(19) United States
(12) Patent Application Publication
(10) Pub. No.: US 2026/0036602 A1
(43) Pub. Date: Feb. 5, 2026

(54) SMART CLUSTER SENSOR SYSTEM AND APPARATUS FOR WATER QUALITY MONITORING

(71) Applicants: THE REGENTS OF THE UNIVERSITY OF CALIFORNIA, Oakland, CA (US); 501CTHREE, Los Angeles, CA (US)

(72) Inventors: Eric M.V. Hoek, Pacific Palisades, CA (US); McKenzie Fowler, El Segundo, CA (US); Jaron Rothkop, Ann Arbor, MI (US); Zachary Finkelstein, Laguna Niguel, CA (US); Jeremy Louie, San Francisco, CA (US); Draco Tong, Palo Alto, CA (US)

(73) Assignees: THE REGENTS OF THE UNIVERSITY OF CALIFORNIA, Oakland, CA (US); 501CTHREE, Los Angeles, CA (US)

(21) Appl. No.: 19/308,198

(22) Filed: Aug. 23, 2025

Related U.S. Application Data
(63) Continuation of application No. PCT/US2024/018809, filed on Mar. 7, 2024.
(60) Provisional application No. 63/488,851, filed on Mar. 7, 2023.

(51) Int. Cl. G01N 35/10; G01N 33/18; G01N 35/00

(52) U.S. Cl. CPC G01N 35/1097; G01N 33/18; G01N 2035/00475; G01N 2035/00891; G01N 2035/0096

(57) ABSTRACT

A smart cluster control unit for water quality monitoring of water filtration systems which, in one embodiment, utilizes multiple sensors, including dissolved oxygen, electrical conductivity, temperature, oxidation reduction potential, and pH sensors. The sensors connect through the control unit which collects and analyzes the information, and to utilize models/relationships to extrapolate additional water quality information. The apparatus allows for the measurement of filter condition by testing the water quality before and after filtration to characterize filter effect and filter performance.

### CROSS-REFERENCE TO RELATED APPLICATIONS

This application claims priority to, and is a 35 U.S.C. § 111(a) continuation of, PCT international application number PCT/US2024/018809 filed on Mar. 7, 2024, incorporated herein by reference in its entirety, which claims priority to, and the benefit of, U.S. provisional patent application Ser. No. 63/488,851 filed on Mar. 7, 2023, incorporated herein by reference in its entirety. Priority is claimed to each of the foregoing applications.

The above-referenced PCT international application was published as PCT International Publication No. WO 2024/186968 A1 on Sep. 12, 2024, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### INCORPORATION-BY-REFERENCE OF COMPUTER PROGRAM APPENDIX

Appendix A referenced herein is a computer program listing in a text file entitled “UC-2022-275-2-LA-US-computer-program-appendix-A.txt” created on Aug. 22, 2025 and having a 43 kb file size. The computer program code, which exceeds 300 lines, is submitted electronically as a computer program listing appendix through Patent Center and is incorporated herein by reference in its entirety.

### BACKGROUND

The technology of this disclosure pertains generally to water quality

### 1. TECHNICAL FIELD

measurement, and more particularly to systems for determining water quality performance in real-time.

### 2. BACKGROUND DISCUSSION

Present methods and systems for indicating filter performance and lifetime are time, volume, pressure and/or usage based. These provide no indication of the actual physicochemical parameters of the water or performance of the filter.

Accordingly, a need exists for an apparatus that assesses in real-time the actual condition of the water filters. The present disclosure fulfills that need and provides additional benefits over existing systems.

### BRIEF SUMMARY

This disclosure describes a smart cluster apparatus for monitoring filter performance in a water system. A number of sensors, preferably including at least dissolved oxygen, electrical conductivity, temperature, oxidation reduction potential, and pH sensors, are coupled to a water quality testing unit, which preferable controls other particulars of the water system to provide the ability for assessing the performance of the water filters in real-time.

In at least one embodiment, the sensors connect through PCBs to a microcontroller, which also connects to other sensors and control outputs in the water system. The embedded microcontroller executes firmware instructions in controlling the timing and steps in collecting and analyzing information acquired from the sensors.

The water quality testing controller unit is able to interoperably utilize the information obtained from its set of sensors, utilized in combination with the settings of the water system. The programming performs temperature compensation, parsing, assigning units, positions, displays, and sends the information to a repository, such as through a wired or wireless network. The technology also describes process steps in performing backend analysis which utilize chemical and physical properties and physical-chemical relationships to extrapolate additional water quality estimations from the various sensor inputs, in response to the state of the water testing system. These include Pourbaix Diagrams for metals of concern and multiple-regression, machine learning (ML) processes.

The entire smart cluster sensor and back-end process steps/ML fulfills the need to monitor the performance of household, commercial, industrial or municipal water filters and monitor basic water parameters which otherwise must be analyzed offline by qualified analytical laboratories. This solution meets that need and combines Internet of Things (IoT) technology to directly convey that information.

The present disclosure describes numerous aspects of an Internet of Things (IoT)-enabled monitoring system for Point-of-Use (POU) applications with these objectives in mind. The disclosed system consists of a sensor package that includes at least water quality parameters of pH, EC, ORP, Dissolved Oxygen (DO), and temperature. The controller of the system provides an embedded controller (e.g., microcontroller, gate array, logic array, discrete logic, application-specific integrated circuit, or combinations thereof) for controlling operations of the system, while analog circuitry is incorporated for signal conditioning.

In addition, the system includes programming for performing data transfer from the controllers and its collected information and assessments to an online repository where collaborators having access can view the information. Preliminary algorithms that utilize reference correlations, statistical analysis techniques, and physical and chemical constants to calculate and predict other water quality parameters such as corrosivity risk have also been developed.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1 is a block diagram of a wireless sensor smart cluster apparatus for water quality monitoring of a filter based water system according to at least one embodiment of the present disclosure.

FIG. 2A and FIG. 2B are a flow diagram of water sensor process flow for the system of FIG. 1, according to at least one embodiment of the present disclosure.

### DETAILED DESCRIPTION

### 1. Introduction

Currently, no smart water quality monitoring solutions are available to inform consumers or small plant operators about real-time water filter performance. Current solutions for indicating filter performance and lifetime are time, volume, pressure and/or usage based, and give no indication of the actual physicochemical parameters of the water or performance of the filter.

As of 2010, safe and accessible drinking water has been explicitly recognized as a human right by the United Nations General Assembly and deemed essential for public health and economic prosperity. Despite this designation, 785 million people lack essential drinking water services globally, and at least two billion people use drinking water sources contaminated with feces. Diseases like cholera, diarrhea, dysentery, hepatitis, typhoid, and polio are linked to such sources of contamination. Despite the risks associated with a lack of essential water services, many drinking water utilities face challenges meeting regulations due to limited water supply, strict budgets, high demands due to population growth, aging infrastructure, and increasingly strict regulations for water quality. Analytical testing and water quality monitoring must be carried out regularly to ensure water is free from biological and chemical contamination. Unfortunately, these methods often take multiple days to conduct, in which case contaminated water may already have had negative impacts on public health.

These challenges can contribute to public water utilities violating regulations established by the EPA, increasing interest in solutions such as point-of-use (POU) filtration, and real-time water quality monitoring. Even if violations are not observed from water utilities there is still potential for contamination between treatment and the tap. This contamination can source back to community water system's distributional networks and property owner's premise plumbing.

Before reaching taps, consumers can install POU drinking water treatment systems in their water supply lines to provide on-site treatment to the water they consume. These systems encompass many treatment technologies such as membranes, filtration, UV disinfection, activated carbon, and so forth. The utility of implementing POU filtration shows in its ability to reduce contamination's acute and chronic health effects. Still, there is little evidence of commercially available technologies to improve the “smartness” of point-of-use water systems in terms of their ability to perform tasks such as monitoring and reporting on water quality. Possible ways to enhance the smartness of point-of-use filters include integrating Internet of Things (IoT) enabled sensor technologies.

### Water Quality Correlations

After investigating the current hardware and software utilized for wireless sensor networks, a key research question to be addressed was to determine the sensors and algorithms that were best suited for smart point-of-use water quality monitoring. Therefore, some of the existing relationships between various sensor parameters and water quality concerns were reviewed to better guide the sensor selection process. Here, we present some of the findings on what water quality information can be related to pH, ORP, DO, and EC

For acute effects from biological contaminants, it is imperative that sufficient disinfection levels are maintained. In drinking water, this is primarily achieved with Chlorine and Chloramine, which previous research has attempted to link to ORP measurements. Experiments have been conducted that investigate the ability of ORP to be a predictor for kill level of organisms including total coliform, E. Coli, and enterococci by measuring the ORP in conjunction with Chlorine dose for wastewater. It was found that the ORP increased with an increase in chlorine added, total chlorine, and free chlorine. However, the regression slopes were relatively low indicating low sensitivity of ORP measurements as a function of each chlorine species. However, the authors highlight that a large change in measured ORP occurs near zero chlorine residual and commented on the effectiveness of controlling chlorine with ORP measurements at low chlorine concentrations, but as the free chlorine concentration increases beyond 1 mg/L the sensitivity of the measurement is reduced.

A pilot-scale application of using ORP and pH to develop a control strategy for chlorination of wastewater was achieved by using significant points occurring on the pH and ORP profiles during chlorination titrations. The results showed that ORP increased from 300 mV to 400 mV when Chlorine solutions were first added, then the ORP profile flattened as NH₂Cl dominated the redox state of the water. At the same time, the pH profile increased due to the hydroxyl ion formed from NaOCl. As more chlorine was added, a second increase in ORP was observed. From this point, Monochloramine is oxidized to Dichloramine, which has a higher redox potential and produces H⁺ leading to a lower solution pH. Once all the Dichloramine was oxidized, free chlorine became available, and the third increase in ORP was observed, indicating the chlorine breakpoint after which viable counting of microorganisms was not observed. The control system implemented could continuously detect the breakpoint and determine the correct chlorine does for inactivating microorganisms in the water. The proposed system ultimately can easily detect the shifting point where dominating chemical species change by monitoring points along with the ORP and pH profiles during chlorination to achieve proper disinfection.

ORP measurements have also been explored for monitoring and controlling water disinfection for the produce washing industry (Suslow, n.d.). ORP ranges between 600 mV and 700 mV show that free-floating decay and spoilage bacteria, as well as pathogenic bacteria such as E. coli or Salmonella species, are killed within 30 seconds. In relation to the pH sensors, lowering the pH raises the percentage of HOCl, and ORP increases to reflect this shift in oxidative potential. Recent research in commercial and model postharvest water systems has shown that, if necessary, ORP criteria can be relied on to determine microbial kill potential across a broad range of water quality. However, it is important to note the limitation of ORP and pH because due to the effects of pH on chlorine speciation, one must use caution in not having a false sense of adequate disinfection rates at high pH's. In general, a ten-fold increase in total or free chlorine concentrations does not result in a corresponding proportional increase in ORP millivolts. Their results showed that good water quality likely results in measurements of 650 mV to 700 mV ORP if the water pH is 6.5 to 7. Lowering the pH to 6.0 raises the ORP as more hypochlorous acid becomes available. Raising the pH to 8.0 lowers the ORP value, as more hypochlorite ions are present. Maintaining constant pH but adding more chlorine raises the ORP to a plateau of about 950 mV. Finally, Myron L Company describes their correlation for predicting free available chlorine using ORP and PH levels. The correlation was obtained from a series of experiments where an exact amount of chlorine in the form of laboratory-grade bleach was added to DI water in a closed system and measuring pH and ORP to create calibration curves for their Myron L Ultrameter II 6PFCE Water Quality Meter. From some of the listed works, it is evidenced that ORP can be used as an effective indicator for disinfection levels in water and has the potential to protect users from potential biological contamination using real-time measurements of ORP in a wireless sensor network.

Other correlations/relationships between the variables measured in the described smart water quality testing system we have discovered utilizing statistical analysis methods and machine learning techniques.

### 2. Smart Cluster Apparatus and Method for Water Quality Measurement

2.1 this Disclosure Describes a Smart Cluster Sensor Method and Apparatus for Water Quality Monitoring which, in One Embodiment, Utilizes Multiple Sensors, Such as Dissolved Oxygen, Electrical Conductivity, Temperature, Oxidation Reduction Potential, and pH Sensors. The Sensor Apparatus is Particularly Well-Suited for Use in a Mobile Water Filtration System.

In use, purified water from a portable water treatment system is tested by filtration system operators for a number of water quality parameters, including: pH, total dissolved solids (TDS), and lead. Additionally, the water is tested for microbiological content by state certified third-party laboratories to ensure the water is clean and safe. Daily tests for lead and microbiological contaminants are conducted and reported on a weekly basis.

The smart cluster sensor apparatus disclosed herein can provide online monitoring for real-time detection of parameters, such as water pressure, flow rate and specific water quality parameters (e.g., pH, EC/TDS, ORP, turbidity, lead, etc.), which could automate, replace and enhance the daily testing (currently performed by operators). The sensor apparatus disclosed herein can be combined with, for example, WiFi and GSM telemetry capabilities to enable both: (a) users to log into an app on their mobile devices (e.g., smartphones) to access current water quality outputs as they are filling their containers with treated water, and (b) staff scientists and engineers to remotely monitor all portable water treatment installations in real-time.

In one embodiment, the sensor apparatus can provide pressure, flow, pH, Electrical Conductivity (EC), and Oxidation-Reduction Potential (ORP) measurements. It should be noted that in certain embodiments turbidity (NTU) and/or lead (Pb) are determined or estimated by incorporating one or more additional sensors depending on the application, or by estimations based on the other information collected. From the combination of pH, EC, Pb, NTU and ORP, one can derive other meaningful water quality parameters such as total dissolved solids/salinity, alkalinity, various saturation indices that establish a water's corrosivity/stability (e.g., a water's propensity for dissolving lead and copper from pipes versus fixing them onto pipe surfaces), lead concentration, chlorine concentration, speciation of common metals (e.g., Fe2+ vs. Fe3+ vs. Fex(OH)y(s)) and potential pathogenic microorganism contamination. In at least one implementation, the sensor apparatus can be embodied as an Internet of Things (IoT) enabled system with, for example, a minimum cluster set comprising pressure, flow, pH, EC and ORP, plus whichever of the sensors are most useful. The cluster sensor apparatus can also be integrated into other types of water filtration systems.

### 2.2 Method

### 2.2.1 Sensor Selection

By way of example and not limitation, Lab grade sensors that use various electrochemical mechanisms were selected for testing of the present disclosure. Primary factors that were considered when selecting the sensors used for the prototype included sensitivity, selectivity, stability, lifetime, response time, pressure tolerance, cost, and size. In addition, the robustness, ease of calibration, compact size, and commercially available plumbing components contributed to the overall selection process for the sensors to be included in the prototype. The complete sensor package includes sensors for pH, EC, DO, ORP, and temperature. Table 1 describes specifications for each of the selected sensors, as utilized in the testing of an embodiment of the present disclosure.

It should be appreciated that for small POU applications, sensors may be utilized which need not be lab grade. Possible inaccuracies/reliable issues may be mitigated by testing these sensors, profiling their characteristics with correction curves passed into the system as calibration curves. In addition, multiple same-sensor clusters of 2-3 sensors may be utilized, whereby any anomaly with one sensor is readily detected as readings are compared between the two in-situ sensors of the same type. This enhances diagnostic capability and allows for averaging and removal of outliers.

A measure of pH is a determination of how acidic or basic water is and is important in water quality because it determines the solubility and biological availability of chemical constituents, nutrients, and heavy metals. Metals tend to be more toxic at lower pH because they are more soluble. The pH sensor measures the hydrogen ion activity in liquids and has a glass membrane where hydrogen ions in the liquid diffuse onto the outer layer of the glass while larger ions remain in solution. The difference of concentration of hydrogen ions on the outside of the probe as compared to inside the probe creates a measurable current that is proportional to the concentration of hydrogen ions in the liquid. In at least one instance the sensor includes an internal double junction, an EXR glass tip, and a body of extruded epoxy, making the sensor suitable for pH measurements of high purity waters and capable of resisting strong acids and bases for contaminated waters.

Electrical conductivity is the measure of a water's ability to pass an electrical current. Substances that conduct electrical current include dissolved salts and other inorganic chemicals. Generally a given water supply will have a relatively constant range for conductivity; therefore, significant changes can provide valid indicators of the pollution of an aquatic resource. Waters with elevated conductivity may have other impaired or altered indicators as well. Inside a typical conductivity sensor, two electrodes are positioned opposite each other. An AC voltage is applied to the electrodes, which results in the cations moving to the negatively charged electrode while the anions move to the positively charged electrode. This conductive state is then conditioned for reading, such as through analog filtering whose output is read by an analog-to-digital converter.

The next sensor parameter selected was dissolved oxygen. At least one type of DO sensor consists of a PTFE membrane, an anode bathed in an electrolyte, and a cathode. The operating principle is based on the oxygen molecules diffusing through the membrane of the sensor at a constant rate. After crossing the membrane, the oxygen molecules then reach the cathode, where they are reduced, and a small voltage is produced, which is conditioned and read by an analog-to-digital converter. Dissolved oxygen is important in drinking water because high DO levels can damage components and systems that are used in drinking water treatment and distribution. Namely, high DO levels can contribute to corrosion in pipes, and if these levels are too low then issues can arise in regard to water taste.

ORP is a measure of the oxidation-reduction potential where oxidation is the loss of electrons and reduction is the gain of electrons. An ORP sensor measures electron activity in a liquid and shows the strength at which electrons are transferred to or from a substance in a liquid. The ORP sensor selected for the testing contains a platinum tip and a 4 molar KCl reference solution. ORP was selected because it, combined with pH and temperature, can be used to estimate free chlorine concentrations, which is an indicator of the presence or absence of disease-causing bacteria and viruses, these are typically the cause of most acute symptoms of waterborne disease or illness. ORP was also selected because it can be used in combination with pH and metal concentrations to plot the equilibrium potential of electrochemical reactions. This is useful in predicting the corrosion risk and speciation of various chemical constituents in aqueous solutions and is incorporated into the back-end software package.

### 2.2.2 Sensor Calibration

To prepare the sensors for data collection of tap water samples, they were first calibrated. For example pH sensor was calibrated using known pH buffer solutions at pH 4.01, pH 7.00, and pH 10.01. The remaining sensors were calibrated using the other known calibration solutions. The conductivity sensor underwent a 2-point calibration with 12,880 μS and 150,000 μS solutions, DO also had a two-point calibration in dry air and a zero dissolved oxygen solution. Lastly, ORP and temperature were calibrated using 225 mV calibration solution and 100° C. boiling water, respectively. It should be appreciated that in at least one embodiment these calibration steps are facilitated by the controller, in situations in which any sensor need to be replaced, whereby the user is guided through this calibration step easily. Specifically, the code of the controller can step the user through the calibration steps for any of the sensors, while the controller registers the results and creates the necessary calibration curves, or equivalent. In at least one embodiment correction values/curves can be stored into the control unit for the specific sensor.

After calibration, the sensor performance was verified by comparing the values of the five sensors against values from a Myron Ultrameter II™ 6PFC^{E}.

### 3. System Architecture

To complete the referenced calibration procedures, the sensors were integrated as part of the embedded system for continuous water quality monitoring of the five parameters. The system can primarily be split into hardware and software components.

### 3.1 Hardware Components

For the hardware, one embodiment of the central measurement system comprises a controller/sequencer and multiple sensors connected into the water system controller.

The water system controller controls the operations and collects water quality information and these values are interoperatively utilized to generate estimations on water quality and the condition of the filters. The control unit also can operate as the gateway to external devices, such as for transferring collected information to an information repository, such as over a wireless (or wired) network.

In a less preferred embodiment, the controller comprises a sequencer which comprises logic and timing circuitry for controlling when and how the sensors get coupled directly into the inlet water supply. This embodiment, however, requires that all analysis of water quality is performed either outside of the controller, or in a separate microcontroller coupled in with the sequencer of the controller.

A practical implementation of the water system controller for portable water systems, is to utilize a microcontroller chip having internal memory registers and firmware instructions as well as analog-to-digital circuit inputs. This microcontroller is preferably mounted on a printed circuit board, which also preferably contains the conditioning circuitry for the sensors, the drive electronics for controlling valve state, as well as state outputs, such as LEDs to indicate what mode the system is in and if any faults have been detected. The water system controller can also include wired/wireless communication circuitry to allow the values of assessed water quality to be sent to an external device which incorporates a display and may include user inputs in controlling operating characteristics. In one embodiment, a microcontroller board is plug-coupled (e.g., piggy-backed) with a board carrying sensor conditioning circuits and other analog circuit functions.

The microcontroller may also be part of an Application-Specific Integrated Circuit (ASIC), which provides all the digital control aspects including external communications, and could also include sensor conditioning circuitry.

One embodiment of the controller is described in greater detail in Section 6.

### 3.2 Firmware Components

The code for testing a prototype was written in python integrated development environment software. The sensor processing firmware initially used pseudo-code for the specific sensors as the starting point for the data collection, and these were modified and customized to provide the desired interoperation between the sensors to generate estimates from the apparatus, and to support real-time data transmission. Once the firmware has booted up, and initialized by an operator, DO, pH, EC, temperature, and ORP data were received from the sensor in periodic intervals (e.g., every second, or other desired period of time). The firmware instructions perform temperature compensation of the registered sensor measurements, parsing this information into integers, assigned units, positioned the information into arrays, and outputting this information for display, such as in a terminal window, or external device during data collection. The sensor measurements and/or estimations can be output to an external device, or saved internally or onto a removable memory device such as onto a micro-SD card, whose circuitry is coupled to the microcontroller. In at least one embodiment, the firmware executes to automatically push various collected measurements and/or estimations to a cloud-based information repository via a wired/wireless network.

The prototype had steps for using an acquisition directory, running a./start command to begin the collection of the water quality information, entering user control inputs, and options for operating conditions, with a command to stop the collection. Table 13 contains an example of source code used for the data acquisition process described above.

In at least one embodiment, this repository is accessed through an external device, such as a mobile device, executing application programming to work with the water system controller.

In different embodiments, the operations of the water system controller can be selected at the controller itself, or through communications from the external device with the water system controller.

Examples of source code used for the sensor information collection and processing are shown in the computer program listing in Appendix A.

After following the listed steps, the collected information can be made accessible to any collaborators with access to the repository account. Features on the repository show each data collection session as a separate .csv file that can be easily exported to for extended processing, such as in excel, R, or SPSS.

Besides the real-time data collection, an additional program was developed for back-end data processing using the ORP and pH sensor data. The back-end algorithms consist of Pourbaix diagrams developed for copper, zinc, iron, and lead at standard temperatures and pressure. These were created using the Nernst equations for redox reactions and acid-base reactions transcribed in Marcel Pourbaix's Atlas of Electrochemical Equilibria in Aqueous Solutions (Pourbaix, 1974).

Pourbaix diagrams, are electrochemical graphs that show possible thermodynamically stable phases for aqueous systems. Using Pourbaix diagrams one can predict the equilibrium states of all the possible reactions between an element, its ions and its solid and gaseous compounds in the presence of water. To construct a Pourbaix diagram a standard chemical potential and ion activities must be assumed for the substances reacting. These diagrams can be read similarly to a standard phase diagram with electrical potential and pH as the axes. The lines of a Pourbaix diagram are developed using the Nernst Equation and show the equilibrium conditions for the species on each side of that line where each species on either side is said to predominate. For a reversible redox reaction with the following equilibrium equation.

$$aA + bB ↔ cC + dD \qquad ( 1 )$$

with an equilibrium constant of

$$K = \frac{{{[ C ]}^{c} [ D ]}^{d}}{{{[ A ]}^{a} [ B ]}^{b}} \qquad ( 2 )$$

the Nernst Equation may be expressed as

$${E}_{H} = {E}^{0} - \frac{RT}{zF} ln K \qquad ( 3 )$$

where E⁰ is the standard potential, R is the universal gas constant, T is temperature in Kelvin, F is Faraday's Constant, z is the ion charge, and K is the equilibrium constant. Substituting the equilibrium constant gives,

$${E}_{H} = {E}^{0} - \frac{RT}{zF} ln \frac{{{[ C ]}^{c} [ D ]}^{d}}{{{[ A ]}^{a} [ B ]}^{b}} \qquad ( 4 )$$

This equation is sometimes simplified to:

$${E}_{H} = {E}^{0} - \frac{{V}_{T} λ}{Z} log \frac{{{[ C ]}^{c} [ D ]}^{d}}{{{[ A ]}^{a} [ B ]}^{b}} \qquad ( 5 )$$

$$where$$

$${V}_{T} = \frac{RT}{F} \approx 0.02569 Volts \qquad ( 6 )$$

which is the thermal voltage or the “Nernst slope” at standard temperature.

The equation can further be simplified using the following.

$$λ = ln ( 10 ) \qquad ( 7 )$$

The equation can be numerically expressed as:

$${E}_{H} = {E}^{0} - \frac{0.05916}{Z} log \frac{{{[ C ]}^{c} [ D ]}^{d}}{{{[ A ]}^{a} [ B ]}^{b}} \qquad ( 8 )$$

Using these equilibrium formulae, one can construct diagrams for various metals of concern in aqueous system. To draw the position of the lines with the Nernst equation, the activity of the chemical species at equilibrium must be defined, for many soluble species the concentrations are assumed 10⁻⁶ M, this convention was followed for the software package developed. Changes in temperature and concentration of solvated ions will affect the equilibrium lines as dictated by the Nernst Equation. To establish diagrams, it must first be noted that there are three types of lines in Pourbaix diagrams: Vertical, horizontal, and sloped. Vertical lines represent reactions where no electrons are exchanged and are acid-base reactions. This will create a boundary line that is vertical at a designated pH value and the reaction involves only protonation/deprotonation. Using the reaction equation

$$aA + bB + {hH}^{+} ↔ cC + dD \qquad ( 9 )$$

and the energy balance is

$$Δ {G}^{0} = - RT ln ( K ) \qquad ( 10 )$$

where the equilibrium constant K takes the form

$$K = \frac{{{[ C ]}^{c} [ D ]}^{d}}{{{{[ A ]}^{a} [ B ]}^{b} [ {H}^{+} ]}^{h}} \qquad ( 11 )$$

Therefore the energy balance can be written as

$$Δ {G}^{0} = - RT ln ( \frac{{{[ C ]}^{c} [ D ]}^{d}}{{{{[ A ]}^{a} [ B ]}^{b} [ {H}^{+} ]}^{h}} ) \qquad ( 12 )$$

Or, in base-10 logarithms

$$Δ {G}^{0} = - RT λ ( log ( \frac{{{[ C ]}^{c} [ D ]}^{d}}{{{{[ A ]}^{a} [ B ]}^{b} [ {H}^{+} ]}^{h}} ) + hpH ) \qquad ( 13 )$$

which may be solved for a particular value of pH. For the establishment of Lead, the vertical lines distinguish between the limits of the domains of predominance for the dissolved substances and the relative stability of the solid substances.

Horizontal lines are created from equilibrium equations for reactions that do not involve H+ or OH− thus the boundary line is independent of pH and the reaction equation may be written as

$$aA + bB + {ze}^{-} ↔ cC + dD \qquad ( 14 )$$

The new energy balance can be expressed as

$$Δ {G}^{0} = RT ln ( \frac{{{[ C ]}^{c} [ D ]}^{d}}{{{[ A ]}^{a} [ B ]}^{b}} ) \qquad ( 15 )$$

and from the electrode potential

$$Δ G = - zFE \qquad ( 16 )$$

The Nernst Equation for the horizontal lines takes the form

$${E}_{h} = {E}^{0} - \frac{{V}_{T}}{z} ln ( \frac{{{[ C ]}^{c} [ D ]}^{d}}{{{[ A ]}^{a} [ B ]}^{b}} ) \qquad ( 17 )$$

or for the base-10 logarithms form

$${E}_{h} = {E}^{0} - \frac{{V}_{T} λ}{z} log ( \frac{{{[ C ]}^{c} [ D ]}^{d}}{{{[ A ]}^{a} [ B ]}^{b}} ) \qquad ( 18 )$$

These may be solved for horizontal lines at specific voltages dependent on the ratio of concentrations for the chemical constituents of each diagram.

Lastly, there are the sloped boundary lines which involve both electrons and H⁺ ions.

$$aA + bB + {hH}^{+} + {ze}^{-} ↔ cC + dD \qquad ( 19 )$$

The Nernst equation becomes in base-10 logarithm form

$${E}_{h} = {E}^{0} - \frac{{V}_{T} λ}{z} log ( \frac{{{[ C ]}^{c} [ D ]}^{d}}{{{[ A ]}^{a} [ B ]}^{b}} + hpH ) \qquad ( 20 )$$

where the h value is the slope of the line. These were then used as the basis for a python script which uses Matplotlib visualization tool to generate the diagram. The pH and ORP value from the sensors are then used to place a point on the Pourbaix diagram. That point is then evaluated using different logic operators and the python Shapely package to determine if the point falls within the immunity, corrosion, or passivation region. The code also returns the predominate species for the aqueous system and their state. This feature was included due to metal contamination from premise plumbing being a primary concern for point of use applications. An example of code for the diagrams is included in Table 14 and contains all the solved Nernst equations for the lines needed to develop the diagrams for lead, copper, iron, and zinc.

### 4. Experimental Design

With the software and hardware components successfully operating the next step in the research process was to experimentally test the sensors to ensure they performed well in drinking water applications and determine if the prototype system could be used for predictive modelling or event detection. Three primary experimental tasks were involved.

### 4.1 Experimental Phase One: Sensor Validation

Reliable sensor measurements are essential for smooth operation of the proposed prototype. If a sensor fails to provide accurate measurements false ideas of the water quality are perpetuated creating potential health risks or leading to suboptimal operation. Thus, the first task for testing the system was to ensure the sensors returned accurate and reasonable results for tap water samples.

A total of 34 tap water samples for testing were collected from a laboratory tap water faucet at the University of California Los Angeles between the hours of 10 AM to 4 PM from the months of August 2021 through October 2021. The samples were collected in 1000 mL volumes in a graduated cylinder. Approximately 100 mL of the sample was immediately tested using the Myron Ultrameter for pH, ORP, EC, and temperature. This process consisted of (a) cleaning the cell cup thoroughly with MilliQ water, (b) turning on the Myron Ultrameter, (c) rinsing the sample cell 3 times with the sample to be tested (d) refilling the sample cell with additional sample, (d) pressing the desired measurement key, and (e) recording the values. The remaining volume of the sample of approximately 900 mL was transferred to a 1000 ml beaker and placed on a hot plate with a magnetic stirrer. The sample was stirred at approximately 750 RPM to mimic the flow of water through an inline pipe system and provide sufficient flow for the DO sensor. The clean and dry sensor sensors were then inserted into the beaker, ensuring that the sensors did not touch the stir bar and had minimal contact with the walls of the 1000 ml beaker. For each sensor run the hotplate was maintained at 25° C.; however, some small temperature variations were observed during testing due to factors such as varying ambient room temperatures and temperature changes in the building pipes throughout the day. Data collection was then initiated using the steps described in the previous software section. The sensors collected one data point every second continuously for 10 minutes to allow sufficient sensors stabilization time. The values for the 10 minute run were then downloaded from the GitHub into an Excel file. The mean value for each sensor was calculated for that sample and recorded in an Excel file with the corresponding Myron Ultrameter value for the same sample. From the remaining ˜900 mL of sample, another 100 mL was set aside and refrigerated for additional laboratory analytical testing.

Once collected this data set was analyzed using two statistical tests recommended for comparing two instruments with continuous data as outlined in NIST Technical Note 2106. In that document the null hypothesis states: H₀: All instruments perform equally well; while the alternative states: H_{A}: Not all instruments perform equally well.

For instrument comparison, the p-value can usually be interpreted as the probability of measuring a disparity as great or greater than that seen, under the assumption that the instruments are truly equivalent. If the p-value is smaller than α=0.05 then the null hypothesis is rejected by the test.

To obtain an appropriate p-value, first the variances between instruments were compared for each measured parameter using an. F-test

$$F = \frac{{s}_{1}^{2}}{{s}_{2}^{2}} \qquad ( 21 )$$

$$where ,$$

$${s}^{2} = \frac{\sum {( x - \overline{x} )}^{2}}{n - 1} \qquad ( 22 )$$

$$and$$

$${s}^{2} = variance$$

$$x = values in data set$$

$$\overline{x} = mean of the data$$

$$n = total number of values$$

After determining if the variances were equal or not, the appropriate test was applied. For equal variance test

$$T = \frac{\overline{x} - \overline{y}}{{s}_{p} \sqrt{\frac{1}{m} + \frac{1}{n}}} \qquad ( 23 )$$

Or in the case of unequal variance

$$T = \frac{\overline{x} - \overline{y}}{\sqrt{\frac{{s}_{x}^{2}}{m} + \frac{{s}_{y}^{2}}{n}}} \qquad ( 24 )$$

$$where ,$$

$$\overline{x} = the mean of the measurements from instrument 1$$

$$\overline{y} = the mean of the measurements for instrument 2$$

$${s}_{x}^{2} = the sample variance for instrument 1$$

$${s}_{y}^{2} = the sample variance for instrument 2$$

The result of this comparison were used to determine if the sensors selected for the prototype gave reasonably accurate results compared to a multiparameter benchtop probe.

### 4.2 Experimental Phase Two: Predictive Model Development

The subsequent phase of the experimental process was the testing of each of the 34 tap water samples for EPA primary and secondary drinking water standards. The purpose of this was to attempt to develop models between the sensor values recorded in phase one and correlate them to other important drinking water values not measured directly by the sensors, but were hypothesized to have some correlation due to physical or chemical phenomena. Turbidity and dissolved organic carbon were chosen because of their potential to be correlated with dissolved oxygen since these parameters are often indicators of microbial activity, which consumes oxygen in water. Various transitions metals were also chosen because metals would presumably contribute to the overall electrical conductivity of a sample, plus ion specific sensors are extremely cost prohibitive so there are economic benefits to attempting to model their concentrations in water. Free chlorine was chosen because it relates highly to the pH and ORP sensor values as discussed in the related work section. Lastly, color was chosen because it is one of the main visual indicators of poor water quality, and therefore a major consumer concern, although it was not expected that any of the sensor values could be used to predict color as they do not feature any optical measurement features. Table 2 lists the parameters tested, the standard method performed for each, and the instrument utilized.

The results obtained from the additional laboratory testing of the samples were matched the sensor values obtained in phase one and added to the database.

The statistical technique chosen for the design of the models was multiple linear regression. This method involves using several explanatory variables to predict the outcome of a response variable. This technique can be used to determine how strong the relationship is between two or more independent variables and one dependent variable. The technique allows one to obtain a predicted value for specific variables. To perform multiple regression several assumptions must be met.

Four principal assumptions were tested to determine the suitability of the data for multivariate regression: (1) there needs to be a linear relationship between the outcome variable and the independent variable, (2) the residuals are normally distributed, (3) there is no multicollinearity, that is the independent variables are not highly correlated with each other, and (4) homoscedasticity must be satisfied meaning the variance of error terms are similar across the values of the independent variables.

A general rule of thumb for multilinear regression is approximately 20 cases per independent variable in the analysis. For testing the linearity scatterplots may be constructed to ensure the data display linear behavior, the data collected over the course of this experiment were screened graphically for any behavior suggesting non-linearity such as exponential, logarithmic or polynomial. To further confirm the linearity Pearson's correlation was calculated to measure the linearity between the data. In statistics Pearson's correlation coefficient is defined as

$$r = \frac{\sum ( {x}_{i} - \overline{x} ) ( {y}_{i} - \overline{y} )}{\sqrt{\sum {( {x}_{i} - \overline{x} )}^{2} \sum {( {y}_{i} - \overline{y} )}^{2}}} . \qquad ( 25 )$$

$$where ,$$

$$r = correlation coefficient$$

$${x}_{i} = values of the x - variable in sample$$

$$\overline{x} = mean of the values of the x - variable$$

$${y}_{i} = values of the y - variable in a sample$$

$$\overline{y} = mean of the values of the y - variable$$

Multicollinearity was also calculated by creating a correlation matrix using Pearson's correlation, via the same method.

The next assumption is the normality of the data, this was tested by calculating and sorting the standard residuals. The frequency values were then calculated, and histograms were generated to observe the distribution. To find the residuals let

$$r = x - {x}_{0} \qquad ( 26 )$$

$$where ,$$

$$r = residual$$

$$x = measured variable$$

$${x}_{0} = predicted variable$$

From this, the standard residuals were obtained via software in the Excel Data Analysis Toolpak. The final assumption is for homoscedasticity, this can be tested for using a statistical test known as the Breusch-Pagan test; it is its own linear regression model. It tests whether the variance of errors from a regression depends on the values of the independent variables. It is a chi-squared test and if the test has a p-value below the threshold of p<0.05 then the null hypothesis of homoskedasticity is rejected. The following equation was used for the chi-squared value

$${χ}^{2} = \sum \frac{{( {O}_{i} - {E}_{i} )}^{2}}{{E}_{i}} \qquad ( 27 )$$

after which the excel function=CHISQ.DIST.RT (χ², df regression) was used to obtain the p-value for each set of regressions. The data was tested to assure it met all the necessary assumptions and multivariate regression was then performed. Because each predicted variable required its own model, nine separate regression analyses were performed for the parameters listed in Table 2. Statistical software typically uses a curve estimation procedure, which was used to produce regression statistics for each of the desired dependent variables. To develop each of the equations it can be said that each dependent variable Y depends on X. For multiple regression

$$Y = {β}_{0} + {β}_{1} {X}_{1} + … + {β}_{ρ} {X}_{ρ} + σ ( Y ) , sd ( Y ) = σ \qquad ( 28 )$$

$$where ,$$

$${β}_{0} = intercept$$

$${β}_{1} … {β}_{ρ} = regression coefficients$$

$$σ = residual standard deviation$$

For this project, there are β_{1,2,3,4,5} which are the coefficients obtained from each sensor value for a given drinking water sample. Given that there are 5 sensors, to obtain each prediction equation a 5×5 system of linear equations was solved. Once the coefficients were determined for each outcome variable the predicted values were compared to the actual values. A regression model deemed to be a good fit ideally results in predicted values close to the observed values. Two statistical tests are appropriate for evaluating the differences between predicted and observed values. The first was the R² value or in the case of multiple linear regression the adjusted R² value. The adjusted R² value incorporates the degrees of freedom of the model and explains the proportions of total variance in the model. The adjusted R² was also obtained using software featured in Excel's data analysis toolpak, however the R² may be calculated directly using Pearson's correlation coefficient in Eq. 25 where,

$${R}^{2} = {( r )}^{2} \qquad ( 29 )$$

and from this.

$${R}_{Adjustecd}^{2} = 1 - \frac{( 1 - {R}^{2} ) ( N - 1 )}{N - p - 1} \qquad ( 30 )$$

$$where ,$$

$${R}^{2} = sample R - square$$

$$p = number of predictors$$

$$N = total sample size$$

In addition to the adjusted R² value, the root mean square error (RMSE) was also calculated. This statistic indicates the absolute fit of the model to the data and is the most important criterion for fit if the main purpose of the model is prediction. The RMSE can be expressed as

$$R M S E = \sqrt{\frac{{\sum }_{i = 1}^{N} {( {x}_{i} - )}^{2}}{N}} \qquad ( 31 )$$

$$where ,$$

$$N = total sample size$$

$${x}_{i} = observed value$$

$$= predicted value$$

Using the above experimental and data analysis techniques the predictive capabilities of the system, and the contribution of each sensor to additional parameters of concern can allow for the utilization of the prototype to be expanded beyond real time monitoring, increasing the systems utility for ensuring clean drinking water.

### 4.3 Experimental Phase Three: Contamination Detection

The final set of experiments were the collection of data for contamination events for future testing of outlier detection algorithms and event detection systems. For these experiments, the sensors were inserted into a 2 L beaker, with water from the laboratory faucet running into the beaker at

$$2.67 \frac{L}{min}$$

through a MasterKleer PVC flexible tubing. The sensors first collected data from the tap water for approximately 12 minutes to establish a baseline before the water was intentionally contaminated with 1000 ppm lead, copper, iron, and zinc solutions made from ACS reagent grade hydrated metal salts, including lead (II) nitrate, iron (III) chloride hexahydrate, copper (II) nitrate trihydrate, and zinc nitrate hexahydrate. The metal solutions were added to the running tap water sequentially in doses of 1 mL-8 mL at a time using a glass pipette. These were chosen because they are soluble in water and dissociate, leading to metal ions in the aqueous solution. The salts undergo the following reactions in water:

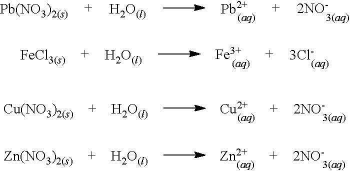

Using a simple dilution calculation, the starting concentration of each contaminant when mixed into the flowing water would be 0.5, 1, 1.5, 2, 2.5, 3, 3.5, 4 ppm. With dilution occurring as tap water flows into the system. A 45 second pause was given between each contaminant dose to allow for values of the system to return to steady state values.

### 5. Results and Discussion

The experimental procedures and data analysis methods described previously serve to gather data on the sensor accuracy/performance, asses the predictive abilities of the system to measure water quality, and determine the sensors' ability to detect contamination events for constituents of concern for point of use applications. The results for each phase of testing the wireless sensor network prototype are presented.

### 5.1 Sensor Verification Results

For the tap water samples which were taken from August 2021 through October 2021, sensor data from the pH, EC, temperature, and ORP sensors were collected and compared to the Myron Ultrameter multiparameter instrument. The comparison results between instruments were plotted for each parameter. Table 3 shows the results of Sensor Statistical F-Tests for Equal or Unequal Variance. Table 4 shows the results of Sensor Statistical p-Tests for comparing instruments.

The results from the F-Test showed unequal variances for all sensors except temperature because F>F_{Critical,one-tail} indicating that the p-test assuming unequal variances must be performed for pH, EC, and ORP sensors. The null hypothesis for comparing instruments states there is no significant difference between the performance of the sensors. To accept the null hypothesis the p-value for the two-tail p test must be less than or equal to α=0.05. Therefore, all the Atlas Scientific sensors, excluding EC (P(T<=t) two-tail >0.05), were found to perform significantly differently from its Myron Ultrameter counterpart.

This result is expected for ORP sensors. The most common problem with ORP measurements for environmental water samples is that readings from various instruments for the same water sample can differ by a significant margin (50-100 mV) even with the same sensor type and electronics, yet the sensors show identical or similar readings in ORP standards. This is explained by the fact that the tap water sampled is expected to be clean by most standards. Therefore, it would likely contain few redox active species present and those that are present have low concentrations, which was confirmed by the results from the Free Chlorine testing of the samples as well. However, in standard solutions the two sensors read the expected values, this is due to the concentration of redox-active species (such as ferricyanide/ferrocyanide in Zobells solution) being much higher. Therefore, it is suggested that historic data be used to help determine the validity of sensors for environmental water samples and inconsistent data between sensors does not always indicate a malfunctioning sensor. As for the discrepancies between the pH values, it is believed that the issue may be due to sensor drift. This is presumed to be the issue due to the similar behavior patterns of the probe with the Atlas pH probe values becoming slightly shifted above the Myron Ultrameter values through the month of October 2021. This indicates more frequent calibration than recommended by the manufacturers may be necessary for long-term sensor deployment. To resolve the discrepancy between the pH probe performance, it would be suggested to collect another set of tap water samples in the future with a recalibration frequency of every 2-4 weeks and determine if the new data allows for acceptance of the null hypothesis. Despite the difference in performance between the sensors and the Myron Ultrameter, the average values for the measured parameters are still in expected ranges for tap water.

### 5.2 Predictive Modeling Results

Information collected from the multiple sensors were also processed using Multivariate Regression Techniques to evaluate the ability to develop predictive models for the parameters listed in Table 5 which shows regression statistics for each predicted variable. The optimized relationships we determined for each parameter were:

$${DOC}_{predicted} = 1.855 - 0.073 DO + 8.52 * 1 {0}^{6} {H}^{+} + 0.00 \; 062 EC + 0.039 Temperature + 0.00046 ORP$$

$$NT {U}_{predicted} = 0.104 - 0.022 DO + 6.35 * 1 {0}^{6} {H}^{+} + 0.00 \; 015 EC + 0.011 Temperature + 0.00019 ORP$$

$$Free {Chlorine}_{predicted} = - 0.2 \; 16 + 0.001 DO - 1.86 * 1 {0}^{6} {H}^{+} + 0.00 \; 013 EC + 0.007 Temperature + 1.39 * 1 {0}^{- 5} ORP$$

$${Copper}_{predicted} = - 0. 07 - 0 \text{.002} DO - 2.34 * 1 {0}^{6} {H}^{+} + 9.74 * 1 {0}^{- 5} EC - 0.003 Temperature + 0.00013 RP$$

$${Lead}_{predicted} = 2.12 * 1 {0}^{- 5} - 4.79 * 1 {0}^{- 8} DO - 1.67 * 1 {0}^{2} {H}^{+} + 3.05 * 1 {0}^{8} EC - 7.72 * 1 {0}^{- 7} Temperature - 1.47 * 1 {0}^{- 8} ORP$$

$${Iron}_{predicted} = 0. 04 - 1.203 * 1 {0}^{- 5} DO - 5.01 * 1 {0}^{2} {H}^{+} - 4.52 * 1 {0}^{- 7} EC - 1.79 * 1 {0}^{- 7} Temperature - 5.62 * 1 {0}^{- 8} ORP$$

$${Zinc}_{predicted} = 0.066 - 0 \text{.0006} DO - 1.32 * 1 {0}^{5} {H}^{+} - 7.05 * 1 {0}^{- 5} EC - 0.001 Temperature + 1.54 * 1 {0}^{- 5} O RP$$

$${Manganese}_{{predicted}^{}} = 3.89 * 1 {0}^{- 5} - 1.54 * 1 {0}^{- 6} DO + 138 \text{.19} {H}^{+} - 3.30 * 1 {0}^{- 8} EC + 1.7 * 1 {0}^{- 7} Temperature - 1.59 * 1 {0}^{- 8} ORP .$$

The above estimations were cross checked using both the Excel data analysis Toolpak and the mldivide function in MATLAB to verify the solutions for each system of linear equations.

The zinc correlations had the best results for both the correlation coefficient and the adjusted coefficient of determination indicating a strong linear relationship for the model. The adjusted coefficient also indicates that 73% of the variation of the zinc values around the mean are explained by the sensor inputs. Of the metal ions, it is observed that the models for lead performed the worst, this is likely due to the low lead concentration in the water leading to negative values obtained for some samples from the ICP-OES, indicating that many of the samples had lead values below the detection limit, which may cause erroneous results. Multiple regression coefficients can also be reduced due to collinearity between the input variables. This was tested using the “correl” function in excel to create a correlation matrix demonstrated in Table 6. Some correlation was observed between pH and EC and DO and ORP, however this should not affect the predictive capabilities of the models, and the correlations are low enough (R²<0.6) to still satisfy the assumption of independence between explanatory variables to meet the model requirements.

### 5.3 Contaminant Dosing Results

Lastly, metal dosing was conducted. In at least one test the last 8 minutes of each run represents the timeframe when the Metal Solutions were introduced. The “isoutlier” function in MATLAB was used to calculate upper and lower thresholds for each sensor. A determination is made if a value is outside either range, and marks values that are determined as outliers with an “x.” The pH results with low values found for outlier detections can be attributed to the acidity of the metal solutions used for dosing which ranged in pH's from approximately 3.5 to 6. Based on the point in time at which outliers were detected it appears that the sensors detected the lowest concentrations for copper followed by zinc, then lead, and finally iron. EC appears to have some false alarms for the zinc and iron contaminations, as values are detected before the metal contamination was introduced into the flowing water. Otherwise, the EC sensors appear to have responded well to the introduction of zinc and sodium chloride as many outliers are detected after the additions of those contaminants. Lastly, many outliers were detected for DO upon the addition of sodium chloride, however they would be expected to decrease because increasing the salinity reduces oxygen solubility indicating bad performance of the DO sensor for anomaly detection.

### 6. System Configuration

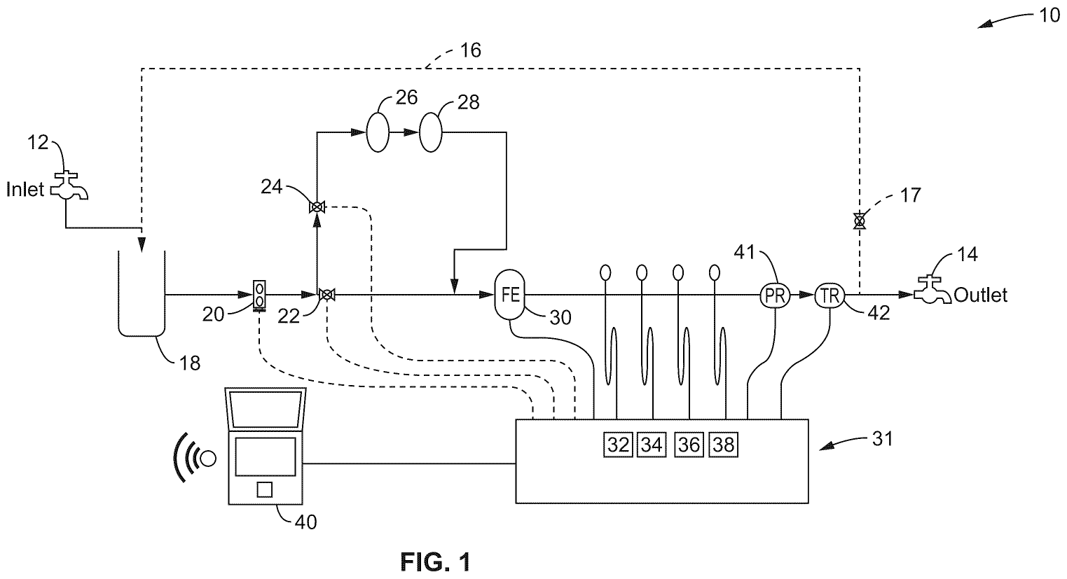

FIG. 1 illustrates an example embodiment 10 of an embodiment of the smart water quality sensing system according to the present disclosure. A water quality control unit 31 is the center of the system, and controls the operation of the numerous elements of the system. The water system is diagrammed having water input 12, traversing through a filter loop to an outlet spigot 14. Inlet water 12 is received in a water tank 18, which is in fluid communication with a pump 20 (e.g., gear pump) that pumps the water through the pipe loop.

In at least one embodiment the operation of the pump is controlled by the user, such as by flipping a switch or pressing a button on the controller, the controller activates the pump and water is passed through the filter loop and its sensor array and is output. In this embodiment, there need not be a valve on the outlet of the unit, although an outlet valve can prevent the water in the system from continuing to drain out of the outlet when the pump is turned off.

In at least one embodiment the operation of the pump is controlled by water quality control unit 31. Preferably, water quality control unit 31 determines if there is flow from the outlet, such as through flow sensor 30, or alternatively (or additionally) utilizing an in-line pressure transducer 41 to determine line pressure. When valve 14 is opened to dispense water, both a flow is detected, and with the pump off the line pressure will drop, whereby controller 31 activates pump 20. In a preferred embodiment, the controller and system as equipped with a pressure sensor 41, sends a pulse-width-modulated (PWM) control signal to the pump, whereby its speed of operation can be adjusted by the controller to maintain a given pressure level over a wide range of water dispensement (outlet flow) rates. The system thus allows the user to set the desired outlet pressure, establish any desired flow rate versus pressure profile. The operation of the pump can also be stopped during other operations, such as when servicing the unit, switching between filter mode and pass-through mode, and so forth. In addition, the pump can be operated in reverse to clear the line back into the reservoir. For example, if the power switch is turned off, then the pump can run shortly in reverse to draw water from the system and bleed off any pressure in the line. Alternatively, if there the water temperature in the system is nearing the freezing point, then the unit automatically reverses the motor to draw all water from the lines and posts a warning. In at least one embodiment configured for cold weather applications the unit is configured with exterior insulation and a heater element whose operation is controlled by controller 31 in response to low temperatures, while equalizing the temperature in the lines by periodically running water alternatively through the filtered path and unfiltered path then through the recycle path.

In another embodiment the pump is configured to only reach a desired pressure level when the unit is powered on. If no water is to be drawn, the unit can be turned off, or an inactive mode selected in which the controller deactivates the pump and enters a low power mode, until the system is activated again.

In one embodiment, the water system is connected to a source of water under sufficient pressure to enable a desired amount of flow through the filters to the outlet. This embodiment does not require a pump while operating in the same manner as the one above with a constant pressure pump, without the need of any intervention by the controller. It should be noted however, that in some applications an automated shut off valve may be coupled to this source of water, such as to block incoming flow if leaks or other issues are detected by the controller.

Valves 22 and 24 control water flow for use and testing. If valve 24 is open and valve 22 is closed, then the water is directed through filtering, exemplified as filters 26, 28, and is subsequently measured through the flow meter 30, reaching water quality control unit 31, exemplified with multiple sensor, and passes across/through the pressure sensor 41 and a temperature sensor 42 before reaching outlet 14.

In the example embodiment, the multiple sensors comprise four water quality sensors exemplified as follows. An Oxidation-Reduction Potential (ORP) Sensor 32 which measures the activity of oxidizers and reducers in an aqueous solution, a Dissolved Oxygen (DO) sensor 34, a pH sensor 36 and an Electrical Conductivity (EC) sensor 38, and temperature sensors 42. In addition to the water quality sensors, information on flow is collected from flow rate sensor 30, and a temperature sensor 42.

The filters 26, 28 may include, but are not limited to, reverse osmosis, kinetic degradation, sediment, remineralization, and granular activated carbon filters.

If valve 24 is closed, then the water flow is directed through the flow meter 30, reaching water quality testing sensor unit 31, exemplified with four sensors 32, 34, 36 and 38 with no filtration step, and passes across/through a temperature sensor 42 before reaching outlet 14. It should be appreciated that the filter testing sensor unit 31 may be configured with additional sensors.

This arrangement allows for the measurement of filter condition by testing the water quality before and after filtration to characterize filter effect and filter performance. After the water quality is tested, the water exits the system through outlet 14. Optionally, the water tested during the testing of the unfiltered water can be passed through recycle loop 16, which may also have the recycle valve 17 so that the water that does not pass through the filters need not be wasted. This is a convenience even when testing the filtered water as it allows the filter test to be performed without the need to output water from the system through outlet 14.

In at least one preferred embodiment valves 22 and 24 are both controlled electronically by the water system controller 31. It should be noted that these valves may be coupled to a single actuator, wherein setting the actuator to a first state closes valve 22 and opens valve 24, and when set in a second state valve 22 opens and valve 24 closes. This ganging of valves can reduce actuator cost.

In at least one preferred embodiment valve 17 is also electronically controlled, which thus allows the system to perform the entirely of the testing at any desired periodic basis without any external user input or manual settings. In at least one embodiment the recycle valve is set by default into recycle mode by the controller when the water output is bypassing the filters, thus assuring that unfiltered water is not distributed. The controller also allows this aspect to be overridden for other testing purposes. The recycle valve also has value to the filtered water flow, so that the water in the system does not sit stagnant for any undue time period.

In at least one embodiment, such as in a lower cost unit, the valves 22, 24 may be manually selectable valves requiring the user to set them during testing. In a preferred embodiment of this configuration, the valves are coupled to sensors (e.g., hall-effect, mechanical switch, optical sensor, potentiometer, rotary position encoder, etc.) so that the state of each valve is read by the water system controller. In this way the controller can commence its test of the non-filtered water in response to detecting that the valves are set in a position for the water to bypass the filters. It will be noted that optional valve 17 would not need to be sensed as it could be left up to the user to whether or not to recycle the water used.

In another embodiment, valves 22 and 24, are entirely manual valves without a position-sensing mechanism attached. In at least one embodiment of this configuration the water system controller is normally taking periodic tests of the filtered water, but upon receiving a user command (e.g., flip a switch/button coupled to controller, or direct input from an external device which communicates with the controller) informing the water system controller to start capturing and processing results for the unfiltered water stream.

The water-filter quality controller unit 31 provides for controlling system functioning, valves and interoperatively reading both water quality and system status, such as temperature, pressure, flow. A plurality of water quality sensors are coupled to a microcontroller which controls when and how the sensors are triggered/read. The information may be directly processed before passing results to an external computing device. A control board and microcomputer are utilized to implement the various relationships/models and provide an ability the transmit the collected data to an online repository to provide for external access. In at least one embodiment, the controller itself is configured with an indelible encoded serial number in its internal flash memory, thus positively identifying the specific unit to field personal, thus assuring data passed to the repository is for the specific unit; and preventing ‘ghosting’ as may be warranted in regard to enforcing water quality testing. While a wireless sensor network is illustrated, it will be appreciated that the sensor network could also be wired.

The system allows for real-time measuring, monitoring, and predicting water quality parameters and filter performance. The smart water filter testing system can be used to predict unexpected additional water quality parameters from the linear combination of each sensor data input using a statistical method of multiple linear regression that is specifically established from a baseline of information also collected through the smart water filter testing system. Predictive models have been developed for the following parameters: turbidity, dissolved organic carbon, free chlorine, copper, lead, iron, zinc, and manganese. This method can also be applied to spot-measurements of water parameters, as captured by reagent-based test strips or discreet sensors. In order to quickly establish a baseline, the data repository may incorporate data collected from other sources, including single-parameter test equipment and reagent-based test strips.

In order to reduce the volume of information, and for the purpose of not transmitting information when water is not flowing through the device, the microcontroller evaluates the differences between sensor measurements over time and only sends data to the remote server when the differences are sufficient to determine that water is flowing through the smart water filter testing system. It should be appreciated that in the preferred embodiments, when a flow sensor 30 is provided the system can readily recognize when water is being output from the unit.

The device can be installed to take measurements across a range of applications, including: household plumbing, commercial plumbing, utility water lines, water fountains and bottle fillers, water treatment systems, water desalinization systems, pour-through water filters, water storage tanks.

### 6.1 Example: Turbidity.

One example of an unexpected prediction made by the sensors is for turbidity. Turbidity is a parameter typically measured using light scattering instruments and can be predicted using the sensor network. These predictive capabilities were developed by the method described.

Samples were obtained from the lab tap water over the course of several months. Each sample was collected from a laboratory tap in 1000 mL volumes, and the sensors were placed in the sample. The sensors measured the water quality over the course of ten minutes and that information evaluated according to the models and relationships described, and converted to a given format, these estimations in the example were sent to a remote data repository as a .csv file and the average value was obtained for the ten minute period. The same sample was then immediately measured for Turbidity using a Hach 2100Q portable turbidimeter. This turbidity value was added to its respective sensor information for each water sample to build up a database to correlate the measured sensor values with the measured turbidity.

These data points were then uploaded to excel, and multiple linear regression was applied to the aggregated data to create the following unique prediction models for turbidity:

$$N T {U}_{p redicted} = 0.104 - 0 \text{.022} DO + 6.35 * 1 {0}^{6} {H}^{+} + 0.00 \; 015 EC + 0.011 Temperature + 0.00019 ORP .$$

This relation was arrived at by “training” the computer to recognize how each measured sensor value affects the output parameter (turbidity) thus providing a relationship that allows predicting future values on turbidity on new sensor input data with reasonable and unexpected accuracy.

Table 7 shows an example of the above relationships used with the sensor inputs collected on tap water sourced from a UCLA laboratory.

### 6.2 Example: Copper

Similarly for Copper, samples were obtained from the lab tap water over the course of several months. Each sample was collected from a laboratory tap in 1000 mL volumes, and the sensors were placed in the sample. The sensors measured the water quality over the course of ten minutes and that data was sent to a remote data repository as a .csv file and the average value was obtained for the ten-minute period. A portion of the 1000 mL was collected in 15 mL centrifuge tubes and was set aside and refrigerated at 6 degrees Celsius for no more than six months. Within those six months the stored samples were tested for metals including copper using an Avio 220 Max Inductively Coupled Plasma Optical Emission Spectrometer. The total copper in the tap water was measured and recorded with its respective sensor data. This data was also uploaded to excel, and multiple linear regression was applied to obtain the following unique relationship, which was used to determine a predicted level of copper. As demonstrated in the following example the predicted vs measured value gives similar results.

$${Copper}_{predicted} = - 0.007 - 0.0 \; 02 DO - 2.34 * 1 {0}^{6} {H}^{+} + 9.74 * {10}^{- 5} EC - 0.003 Temperature + 0.00013 ORP$$

Table 8 shows an example of copper estimation.

### 6.3 Example: Dissolved Organic Carbon (DOC)

Dissolved organic carbon data was obtained to develop another predictive model. Samples were obtained from the lab tap water over the course of several months. Each sample was collected from a laboratory tap in 1000 mL volumes, and the sensors were placed in the sample. The sensors measured the water quality over the course of ten minutes and that data was sent to a remote data repository as a .csv file and the average value was obtained for the ten-minute period. To measure the amount of dissolved organic carbon the samples were stored and within 28 days were filtered and analyzed using a device called a Shimadzu TOC-L. This information was exported from the device and matched with its corresponding sample information. Once matched these could also be used to develop the following multiple linear regression model.

$${DOC}_{p redicted} = 1.855 - 0.0 \; 73 DO + 8.52 * 1 {0}^{6} {H}^{+} + 0.00 \; 062 EC + 0.039 Temperature + 0.00046 RP$$

As in the previous examples, the model provides a similar predicted value when compared to the measured value.

Table 9 shows an example of DOC estimations.

### Example: Test Strip Integration into the Disclosed Testing System

In conjunction with the smart water quality sensing system, an embodiment of the technology of this disclosure includes a “a smart water test strip”. In at least one embodiment, the test strips are single use, reagent based, and sourced from an outside manufacturer. The strips detect the presence of certain chemicals in response to which they change color, with the extent of the color change being indicative of the concentration of that specific contaminant.

Typical test strips require the user to compare the color results on their strip to a color chart provided by the manufacturer which can lead to human error due to individual differences in the perception of color, whereby the user is required to guess at the best interpretation for colors that fall between the color samples on the provided color charts. Due to the variability introduced by different perceptions, test repeatability and reliability is compromised.

In the present disclosure these tests are integrated into the system using image processing components and software that first captures an image of the test strip and then interprets the results which includes applying color correction and noise reduction. Following these steps, the system then obtain the center pixel value, and the most dominant pixel value for the image and retrieves the RGB, hex, and lab values within the test strip result. Once the color value is obtained, a variety of statistical and color difference operations are performed to interpret the result and return a precise and repeatable interpolated value for each contaminant.

Some operations performed for the color difference include Delta E which quantifies the difference between two colors that appear on a screen and original color of standard input. Delta E is measured on a scale from 0 to 100, where 0 is less color difference, and 100 indicates complete distortion. It accounts for the lightness difference, the redness or greyness, and the blueness-yellowness difference between the sample and standard color. The mathematical expression for Delta E is as follows:

$$Δ {E}_{a b}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} \qquad ( 32 )$$

Another formula used is the Matching Factor obtained from the following equation:

$${MF}_{RGB} ( i ) = 1 - \frac{Δ R ( i ) + Δ G ( i ) + Δ B ( i )}{255 * 3} \qquad ( 33 )$$

which was developed for use in processing urinalysis test strip results using smart phones with a result of 1 indicating a perfect match and 0 indicating that the colors are opposite each other.

Once the information is returned, the results are processed and can be shared to a central database where information from both the test strips and the sensor networks can be compiled for further analysis. Furthermore, the test strip results serve as a calibration means for evaluating sensor drift and establishing a baseline of water quality that is used to train the machine learning models that make predictions on additional water quality parameters.

### 7. Water Assessment Processing

### 7.1 Introduction

The firmware of the controller collects data from the connected sensors in response to the settings of the valves and the system operating parameters such as flow, pressure and temperature, both before and after filtration.

The firmware associated with the device generates predictions, estimations, alerts, and recommendations based on the water quality of the water passing through the device. This includes information on disinfectants, inorganic chemicals, microorganisms, organic chemicals, filter performance, and contamination events. In at least one embodiment, the assessments generated from the water system control unit may receive further processing, such as trends as well as analyzing trends across a collection of such water systems.

### 7.2 Machine Learning

Toward making proper predictions/estimations the present disclosure is configured to utilize Machine Learning (ML) to generate additional predictions/estimations on water quality contaminants, measured and predicted water quality, and generating alerts on filter performance as well as performing outlier detection.

### 7.3 Predictive Methodologies

A number of methods can be utilized in arriving at sufficient precision, such as the following. (a) Regression and classification models for prediction/estimates on additional water quality parameters. (b) Linear/Multiple Linear regression. (c) Recommendation engine models for recommending information, services, websites, actions to end user. (d) Anomaly Detection and Time Series Analysis for evaluating filter performance and contamination events. (e) Anomaly Detection. (f) Corrosion Risk assessment model.

### 7.4 Process for Predictive Method

The unit is activated to collect water quality information through the sensors. Real-time measurements are thus obtained from the device sensors on parameters including, but not limited to pH, temperature, dissolved oxygen, and turbidity. The controller may fully process the information, or perform preprocessing. Then a communication link is established to an external device, such as a cloud-based server. The collected/analyzed information is communicated to the external repository for centralized storage and processing.

The preprocessing of the data cleans and organizes initial (raw) sensor measurements, including addressing missing or erroneous values. Apply the appropriate conditioning methods are applied with consideration to model accuracy, efficiency, and other optimization considerations. From the base set of sensed water quality characteristics, additional water quality metrics can be generated by the system, including recommendations, anomaly alerts, corrosion risk, and filter performance data extrapolated from the collected data. The predicted water quality results are aggregated into a packet (e.g., dataset). The system then organizes results into interpretable results, and establishes secure communication protocols for transmitting results to the end user. The results are formatted and presented in a manner which is readily understandable to the end user. A notification system is implemented to alert the end user of newly available water quality information, warning, and assessments. The integration of the described algorithms provides a novel and sophisticated solution for point of use water quality monitoring and management within the apparatus described.

### 8. Test Strip Technology—Applied Example/Technology Details

In this embodiment a mobile device (e.g., smartphone) was adapted to quantitatively analyze commercially available test strips to obtain fast pH measurements using colorimetric detection. In at least one simplified embodiment a mobile device containing an image sensor, is utilized to capture image(s) of the test strip, with the mobile device executing a specific application program designed for this smart water quality test device. That application program scans the strip and returns an exact result based on the color change observed on the test strip when the reagent pad interacts with a liquid. Due to the connective features when using a mobile device, the results obtained can be readily shared using different communication formats and over cloud based services. These results can then be utilized for quality control, calibration, field measurements, and so forth.

A demonstration of the technology utilized in a laboratory setting is as follows.

To implement this technology first requires a calibration curve for the test strip utilized in the test. For this example, a generic 16-in-1 multiparameter test strip was used.

### 8.1 Example Test Strip Processing Procedure.

(1) The test strip is immersed into pH buffer solutions of pH 4.7 and 10 according to manufacturer instructions.
(2) The test strips develop for a sufficient period of time (e.g., 15 seconds) and are placed under an even lighting source (e.g., a ring light), such as set at 4500 Kelvin color temperature level, adjacent to a 24 standard color calibration card and then an image of this combination is captured. The light should provide constant illumination levels, although certain flash units of an image capture device (e.g., mobile device) may provide sufficient even lighting.
(3) The images are then color corrected using a white balance that is set to the white reference on the 24 color calibration standard card. This is to account for differences in lighting.
(4) Once color corrected, the programming performs cropping to isolate the desired reagent pad in the multiparameter test strip. It should be appreciated that the programming can rapidly, repeatably, and accurately perform this cropping step.
(5) After cropping, the images are electronically stored and processed according to the present disclosure using image processing instructions that perform the following steps a through d. An example set of python instructions, called returncolors.py, for performing steps a through d is shown in Table 15.
a. Applying a low-pass filter, such as a Gaussian blur, to reduce random noise in the image.
b. Extracting the dominant color from the image using the colorthief module.
c. Returning the dominant color as an RGB value, which is converted to color spaces including CIELAB, grayscale intensity, and HEX code.
d. Creating a data frame with the color results and exporting, such as using a *.csv file.
(6) Sample results obtained in step 5 are shown in Table 10.
(7) Determining a trendline, from the results of step 5, for the calibration curve by determining a line of optimum fit.
(8) Generating a color gradient in response to selecting a range of pH values into the trendline curves.
(9) The steps above establish the basis for determining where along the trendline/gradient that the new test strip information lies. With this process, a new test strip can be tested in a liquid (in this case tap water) and the image is then processed and based on the returned RGB values, whereby a best pH match can be determined using the following formulas for RGB or LAB values. Incorporation of the HSV color space is also currently in development. With Eq. 34, values closer to one are the best match, while with Eq. 35 values closer to zero indicate the best match.

$${MF}_{RGB} ( i ) = 1 - ( \frac{Δ R ( i ) + Δ G ( i ) + Δ B ( i )}{255 * 3} ) \qquad ( 34 )$$

$$Δ {E}_{a b}^{*} = \sqrt{{( {L}_{2}^{*} - {L}_{1}^{*} )}^{⋀} 2 + {( {a}_{2}^{*} - {a}_{1}^{*} )}^{⋀} 2 + {( {b}_{2}^{*} - {b}_{1}^{*} )}^{⋀} 2} \qquad ( 35 )$$

(10) The following is an example of computing the pH value of a new input image for a test strip tested in tap water:
a. A test strip reagent pad was processed using the returncolors.py script.
b. Returncolors.py (Table 15) was used to obtain the dominant colors as shown in Table 11.
c. A best match was found by using Eq. 34 along the gradient values and finding the minimum MF_{RGB} value, and the maximum ΔE values as shown in Table 12.
d. It should be noted that this model under predicts the measured pH value of 7.4 at a value of 6.5, however, this is as a result of issues with test strip manufacturer in utilizing this technology with. In at least one embodiment, the models can be adjusted for suiting different types and manufacturers of the test strips. This is important as the present disclosure provides more accurate results than relying on human sight-but this requires more accuracy, or at least repeatability, in the test strips. Therefore, the models utilized may be adjusted for new test strip results that have not been tested, thus there may be changes in accuracy or the model may be adjusted, however the same general concept and steps apply.

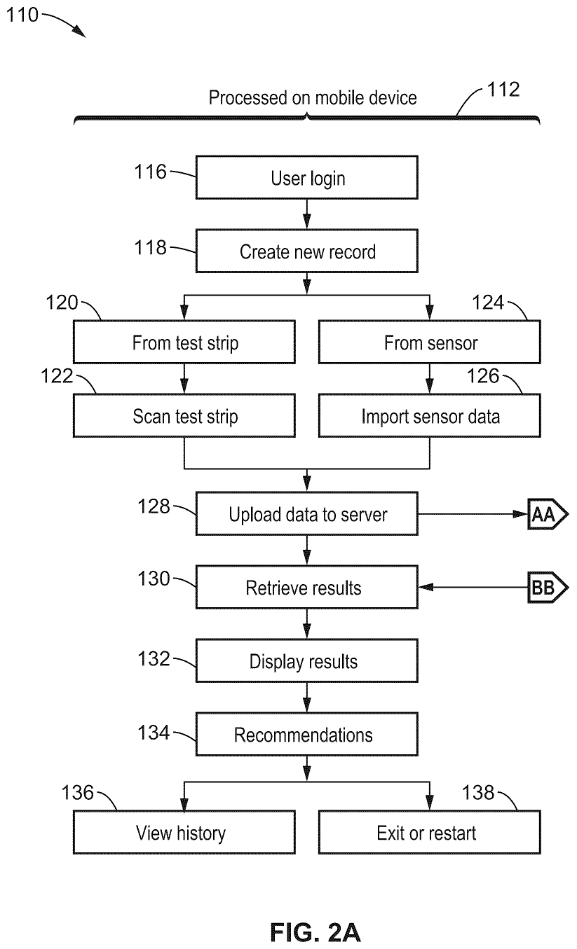

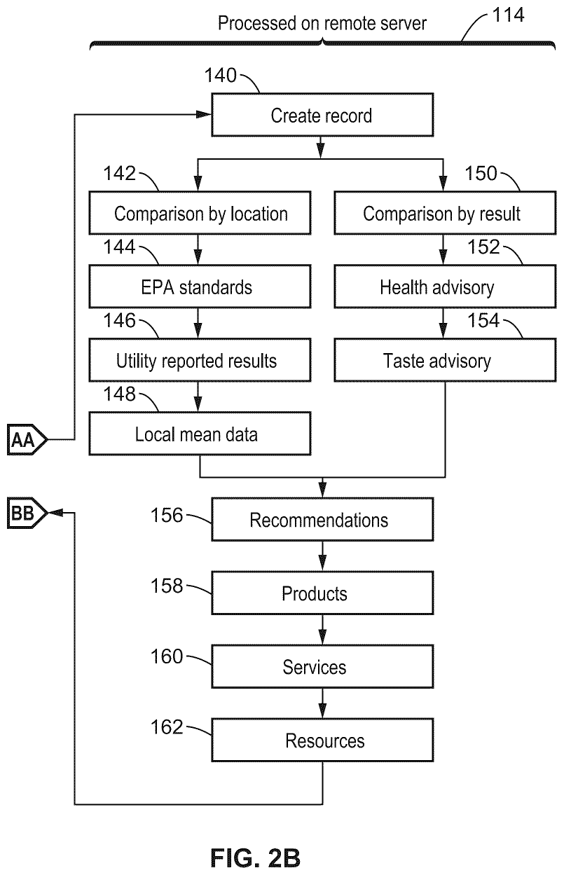

FIG. 2A and FIG. 2B illustrate an embodiment of processing steps for the processing described above. The processing shown in those figures proceeds as follows:

Operations 116 through 128, as seen in FIG. 2A, are performed on an application executed from mobile device performing an application program configured to interoperate with the Smart Cluster Sensor System.
Step 116: User Login.
a. The user logs into the application using a unique ID and password.
b. Login may use 3rd-party user verification.
c. Login may use a two-step authorization.
d. New users are prompted to create a unique ID and password.
Step 118: Create new record.
a. The user is presented with the option of creating a new record by
importing data from the sensor or scanning a test strip.
Step 120: From test strip.
a. The user is provided with directions for testing using a reagent test strip.
Step 122: Scan test strip.
a. An image is captures of the test strip.
Step 124: Obtaining information from Smart Sensor Controller.
a. The user is prompted to connect the mobile device upon which the image was captured, and that device connects (e.g., a wired, or more preferably a wireless connectivity) with the Smart Sensor Controller of the present disclosure.
Step 126: Importing sensor data.
a. Once the connection is made, then transferring information from the Smart Sensor Controller into the mobile device.
Step 128: Upload data to the server.
a. Performing a connectivity check with the server.
b. If there is connectivity with the server, all new records are transferred.
c. If there is insufficient connectivity, the user is informed and asked to try again at another time.

Operations 140 through 162 as seen in FIG. 2B are performed on a remote server.
Step 140: Create a record.
a. A new data record associated with the user's unique ID and sensor serial number is created
b. The data record includes the raw data captured from the sensor, or the image captured of the test strip
c. For test strips, the raw image is color-corrected and the appropriate test values are determined based on the color space of the test strip reagent area.
d. For sensor data, the raw data is indexed against internal lookup tables to determine the test values.
Step 142: Comparison by location.
a. The test values are compared to other records by associated location, which may be the city, state, province, or zip code where the record was created.
Step 144: EPA standards.
a. The test values are compared to a database of state and federal water quality standards.
Step 146: Utility reported results.
a. The test values are compared to a database of results reported to authorities by local water utilities.
Step 148: Local mean data.
a. The values are compared to a database of locally recorded results from other users.
Step 150: Comparison by result.
a. The test record (step 140) is compared to internally established values.
Step 152: Health advisory.
a. The test values are compared to internally established values for safe drinking water levels resulting in a user advisory such as ‘safe’, ‘caution’, ‘warning’, or safety score.
Step 154: Taste advisory.
a. The test values are compared to internally established values for quality of taste, resulting in a user advisory, such as ‘good’, ‘fair’, ‘poor’, or a numerical taste score.
Step 156: Recommendations.
a. By indexing the by-location and by-result values, recommendations can be returned to the user.
Step 158: Products.
a. A list of products is indexed based on the health and taste advisory results.
b. The product recommendations may include websites, social media sites, or phone numbers.
Step 160: Services.
a. A list of local service providers is indexed based on the user's location.
b. The service recommendations may include websites, social media sites, or phone numbers.
Step 162: Resources.
a. A list of local resources, such as water distribution points, is indexed based on the user's location.
b. The resources may include websites, social media sites, or phone numbers.
Steps 130 through 138 are performed on the user's mobile device.
Step 130: Retrieve results.
a. Results for the most recent data record are retrieved from the remote server.
Step 132: Display results.
a. Interpretation of the sensor data is displayed for the user as test results.
b. Results that exceed water quality standards are highlighted for the user.
Step 134: Recommendations.
a. Recommendations for products, services, and resources are displayed for the user.
b. The display may include links to external resources, such as websites, social media sites, and phone numbers.
Step 136: View history.
a. The user is provided with the option to view previous test records.
Step 138: Exit or restart.
a. The user is provided the option to exit the application or return to the start.

### 9. Conclusions

The design and prototype described present a smart cluster system for water quality, filter quality, monitoring for point of use applications. The system architecture can be built on a small microprocessor and includes firmware that allows for remote access of collected sensor information and estimations for users and operators. The experimental results were found to demonstrate the ability of the system to predict other primary and secondary drinking water standards with satisfactory Root-Mean-Square-Error (RMSE) values for the dataset that was tested, as well as recognize outliers caused by contamination events. The project could be expanded upon by integrating the predictive models into automated data processing scripts and generally making the system more autonomous. The proposed algorithms can be improved upon with more robust data analysis and machine learning techniques due to the complexity of a data set by extending the number of sensors being utilized. Future work includes retrofitting the sensor prototype into mobile water filtration units deployed in areas with known water quality issues and developing an application program for a mobile device which interoperates with the smart water quality sensing system which alerts users of water quality issue when they are detected. Overall, the viability of the system is promising for combining many of the advantages of wireless sensor networks for remote monitoring of drinking water.

### 10. General Scope of Embodiments

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure(s) algorithm(s), step(s), operation(s), formula (e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple implementations of the technology which include, but are not limited to, the following:

A water filtration apparatus, comprising: (a) a water filtration loop in fluid communication between a fluid reservoir for receiving water at an inlet, and an outlet; (b) at least one water filter connected within said water filtration loop; (c) valves connected along said water filtration loop to control the flow of water through the filters; (d) a controller configured for controlling the on/off state of said valves when determining the condition of the water filters; (e) wherein said valves can be switched between a first state in which water bypasses said at least one water filter in traversing from inlet to outlet, and a second state in which water passes through said at least one water filter; (f) a plurality of water quality sensors on said controller for collecting water quality information, comprising: a flow sensor, an oxidation-reduction potential (ORP) sensor, dissolved oxygen (DO) sensor, a pH sensor. an electrical conductivity (EC) sensor, and a temperature sensor; and (g) wherein said controller executes firmware instructions from its microcontroller, for performing steps comprising: (g)(i) switching said valves between said first state and said second state, or detecting that manual or automated switching has occurred between said first state and said second state, whereby said controller is configured to collect and assess water quality comprising steps: (g)(i)(A) comparing the quality of water which passes through the filters with water that has not passed through the filters to determine the replacement interval of the filter media as determined by the combination of sensor values; (g)(i)(B) comparing the quality of water which passes through the filters with water that has not passed through the filters to determine the efficacy of the filter media at removing specific contaminants present in the water as determined by the combination of sensor values and comparison to stored reference values; (g)(i)(C) comparing the quality of water which passes through the filters with water that has not passed through the filters to determine the type of contaminants present in the water by comparing the combination of sensor values to stored reference values; and (g)(i)(D) comparing the quality of water which has not passed through the filters to measurements from other devices located in the same water system to determine the source of contamination in the water.

A water filtration apparatus, comprising: (a) a water filtration loop in fluid communication between a fluid reservoir for receiving water at an inlet, and an outlet; (b) at least one water filter connected within said water filtration loop; (c) valves connected along said water filtration loop to control the flow of water through the filters, wherein each said valve is a manual valve coupled to a position sensing device to communicate the state of said one or more valves back to the controller, allowing said controller to automatically perform its operations based on positioning of the valves; (d) a controller configured for controlling the on/off state of said valves when determining the condition of the water filters; (e) wherein said valves can be switched between a first state in which water bypasses said at least one water filter in traversing from inlet to outlet, and a second state in which water passes through said at least one water filter; (f) a plurality of water quality sensors on said controller for collecting water quality information, comprising: a flow sensor, an oxidation-reduction potential (ORP) sensor, dissolved oxygen (DO) sensor, a pH sensor. an electrical conductivity (EC) sensor, and a temperature sensor; and (g) wherein said controller executes firmware instructions from its microcontroller, for performing steps comprising: (g)(i) responding to the detection of said valves being switched between said first state and said second state, said controller is configured to collect and assess water quality comprising steps: (g)(i)(A) comparing the quality of water which passes through the filters with water that has not passed through the filters to determine the replacement interval of the filter media as determined by the combination of sensor values; (g)(i)(B) comparing the quality of water which passes through the filters with water that has not passed through the filters to determine the efficacy of the filter media at removing specific contaminants present in the water as determined by the combination of sensor values and comparison to stored reference values; (g)(i)(C) comparing the quality of water which passes through the filters with water that has not passed through the filters to determine the type of contaminants present in the water by comparing the combination of sensor values to stored reference values; and (g)(i)(D) comparing the quality of water which has not passed through the filters to measurements from other devices located in the same water system to determine the source of contamination in the water.

A water filtration apparatus, comprising: (a) a water filtration loop in fluid communication between a fluid reservoir for receiving water at an inlet, and an outlet; (b) at least one water filter connected within said water filtration loop; (c) valves connected along said water filtration loop to control the flow of water through the filters, wherein said valves are coupled to an actuator coupled to said controller which switches these valves automatically in performing filter testing; (d) a controller configured for controlling the on/off state of said valves when determining the condition of the water filters; (e) wherein said valves are switched between a first state in which water bypasses said at least one water filter in traversing from inlet to outlet, and a second state in which water passes through said at least one water filter; (f) a plurality of water quality sensors on said controller for collecting water quality information, comprising: a flow sensor, an oxidation-reduction potential (ORP) sensor, dissolved oxygen (DO) sensor, a pH sensor. an electrical conductivity (EC) sensor, and a temperature sensor; and (g) wherein said controller executes firmware instructions from its microcontroller, for performing steps comprising: (g)(i) switching said valves between said first state and said second state, upon which said controller is configured to collect and assess water quality comprising steps: (g)(i)(A) comparing the quality of water which passes through the filters with water that has not passed through the filters to determine the replacement interval of the filter media as determined by the combination of sensor values; (g)(i)(B) comparing the quality of water which passes through the filters with water that has not passed through the filters to determine the efficacy of the filter media at removing specific contaminants present in the water as determined by the combination of sensor values and comparison to stored reference values; (g)(i)(C) comparing the quality of water which passes through the filters with water that has not passed through the filters to determine the type of contaminants present in the water by comparing the combination of sensor values to stored reference values; and (g)(i)(D) comparing the quality of water which has not passed through the filters to measurements from other devices located in the same water system to determine the source of contamination in the water.

A system for measuring water quality, the system comprising: a plurality of sensors; said sensors comprising at least one pH sensor, at least one oxidation-reduction potential (ORP) sensor, at least one electrical conductivity (EC) sensor, and at least one dissolved organic carbon (DO) sensor; each said sensor configured to generate an output signal when the sensor is exposed to water; a prediction engine coupled to said sensors and configured to generate a predicted output based on an input; said input to the prediction engine comprising output signals from the sensors corresponding to pH, ORP, EC and DO; said predicted output comprising characteristics of said water selected from the group consisting of dissolved organic carbon (DOC) content, turbidity, bacterial contamination, free chlorine content, copper content, lead content, iron content, zinc content, and manganese content; wherein said water characteristics are predicted without direct measurement of organic, particulate or metal content of said water.

The apparatus or method or system of any preceding implementation, wherein said at least one water filter is selected from the group of filter types consisting of reverse osmosis, kinetic degradation, sediment, remineralization, and granular activated carbon filters.

The apparatus or method or system of any preceding implementation, further comprising a recycle flow path in the water system, whereby water may be redirected from the outlet back to the fluid reservoir for receiving water at the inlet.

The apparatus or method or system of any preceding implementation, further comprising a communications circuit coupled to said microcontroller, to allow said controller to communicate with external devices and/or displays.

The apparatus or method or system of any preceding implementation, further comprising a communications circuit coupled to said microcontroller, to allow said controller to store collected information from each test session into a repository allowing access by collaborators.

The apparatus or method or system of any preceding implementation, wherein said controller further comprises analog conditioning circuitry for one or more of the sensors.

The apparatus or method or system of any preceding implementation, wherein said valves are coupled to an actuator allowing said controller to switch these valves automatically in performing the filter testing.

The apparatus or method or system of any preceding implementation, wherein said valves are manual valves.

The apparatus or method or system of any preceding implementation, wherein a valve position sensing device is coupled to one or more of said valves to communicate the state of said one or more valves back to the controller, allowing said controller to automatically perform its operations based on positioning of the valves.

The apparatus or method or system of any preceding implementation, further comprising calibrating the sensors prior to collection of water samples.

The apparatus or method or system of any preceding implementation, wherein said prediction engine comprises: (a) a processor; and (b) a non-transitory memory storing instructions executable by the processor; (c) wherein said instructions, when executed by the processor, configure the predictive engine to predict said characteristics of said water from said input.

The apparatus or method or system of any preceding implementation, wherein said instructions comprise machine learning processes.

Each and every example, implementation or embodiment of the technology described herein, as well as any aspect, component, or element of any example, implementation or embodiment described herein, and any combination of aspects, components or elements of any example, implementation or embodiment described herein.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has . . . a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

All text in a drawing figure is hereby incorporated into the disclosure and is to be treated as part of the written description of the drawing figure.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Example Sensor Specifications

| Probe Type | pH | | ORP | | DO | | EC | |
|---|---|---|---|---|---|---|---|---|
| Range | 0-14 | | +/−2000 | mV | 0-100 | mg/L | 0.07-50,000 | uS/cm |
| Resolution | +/−0.001 | | — | | — | | — | |
| Accuracy | +/−0.002 | | +/−1 | mV | +/−0.05 | mg/L | +/−2% | |
| Response Time | 95% in 1s | | 95% in 1s | | ~ 0.3 mg/L/per sec | | 90% in 1s | |
| Temperature Range | −5-99° | C. | 1-99° | C. | 1-60° | C. | 1-110° | C. |
| Max Pressure | 100 | PSI | 100 | PSI | 500 | PSI | 500 | PSI |
| Max Depth | 70 m (230 ft) | | 70 m (230 ft) | | 352 m (1,157 ft) | | 352 | m |
| Internal Temperature Sensor | No | | No | | No | | No | |
| Time Before Recalibration | ~1 | Year | ~1 | Year | ~1 | Year | ~10 | Years |
| Life Expectancy | ~2.5 | Years | ~2 | Years | ~4 | Years | ~10 | Years |

TABLE 2
Primary and Secondary Drinking Water Parameters Tested

| Parameter | Method | Instrument * |
|---|---|---|
| Turbidity | USEPA Method 180.1 | 1 |
| Color | ASTM D1209 | 1 |
| Free Chlorine | Hach Method 8021 | 2 |
| Dissolved Organic Carbon | SM 5310B | 3 |
| Zinc | USEPA Method 6010D | 4 |
| Iron | USEPA Method 6010D | 4 |
| Copper | USEPA Method 6010D | 4 |
| Lead | USEPA Method 6010D | 4 |
| Manganese | USEPA Method 6010D | 4 |

* 1 - Hach 2100P Handheld Turbidity Meter
2 - Beckman DU-530 UV-VIS spectrophotometer
3 - Simadszu TOC-L series
4 - Avio 220 Max ICP Optical Emission Spectroscopy

TABLE 3
Sensor Statistical F-Tests for Equal or Unequal Variance

| | Test | | | | | | | |
|---|---|---|---|---|---|---|---|---|
| | pH | | Temp | | EC | | ORP | |
| | Brand* | | | | | | | |
| | A | B | A | B | A | B | A | B |
| Mean | 7.8 | 7.7 | 23.2 | 22.5 | 545.1 | 537.9 | 461.6 | 311.8 |
| Variance | 0.040 | 0.005 | 0.903 | 1.231 | 736.899 | 742.779 | 14287.019 | 1699.621 |
| Observations | 35 | 35 | 35 | 35 | 34 | 34 | 36 | 36 |
| df | 34 | 34 | 34 | 34 | 33 | 33 | 35 | 35 |
| F | 7.656 | — | — | 1.364 | — | 1.008 | 8.406 | — |
| P(F <= f) | 2.148E−08 | — | — | 1.848E−01 | — | 4.910E−01 | 3.714E−09 | — |
| one-tail | | | | | | | | |
| F Critical | 1.772E+00 | — | — | 1.772E+00 | — | 1.788E+00 | 1.757E+00 | — |
| one-tail | | | | | | | | |

*A—Atlas Scientific, B—Myron Ultrameter

TABLE 4
Sensor Statistical p-Tests for Comparing Instruments

| | Test | | | | | | | |
|---|---|---|---|---|---|---|---|---|
| | pH | | Temp | | pH | | ORP | |
| | Brand¹ | | | | | | | |
| | A | B | A | B | A | B | A | B |
| Mean | 7.77 | 7.69 | 23.19 | 22.53 | 545.06 | 537.91 | 461.60 | 311.75 |
| Variance | 0.04039 | 0.00528 | 0.90257 | 1.23141 | 736.89898 | 742.77865 | 14287.01942 | 1699.62143 |
| Observations | 35 | 35 | 35 | 35 | 34 | 34 | 36 | 36 |
| HMD² | — | — | — | 1.06699 | — | — | — | — |
| HMD² | 0 | — | — | 0 | — | 0 | 0 | — |
| df | 43 | — | — | 68 | — | 66 | 43 | — |
| t Stat | 2.03 | — | — | −2.67 | — | −1.08 | 7.11 | — |
| P(T <= t) | 2.44E−02 | — | — | 4.68E−03 | — | 1.41E−01 | 4.44E−09 | — |
| one-tail | | | | | | | | |
| t Critical | 1.68 | — | — | 1.67 | — | 1.67 | 1.68 | — |
| one-tail | | | | | | | | |
| P(T <= t) | 4.89E−02 | — | — | 9.37E−03 | — | 0.282 | 8.89E−09 | — |
| two-tail | | | | | | | | |
| t Critical | 2.0167 | — | — | 1.9955 | — | 1.9966 | 2.0167 | — |
| two-tail | | | | | | | | |

¹A—Atlas Scientific, B—Myron Ultrameter
²HMD = Hypothesized Mean Difference

TABLE 5
Regression Statistics for Sensor Modelling

| | | | Free | | | | | |
|---|---|---|---|---|---|---|---|---|
| | DOC | Turbidity | Chlorine | Copper | Lead | Zinc | Manganese | Iron |
| Multiple R | 0.47 | 0.44 | 0.57 | 0.71 | 0.51 | 0.88 | 0.78 | 0.81037973 |
| R Square | 0.22 | 0.19 | 0.32 | 0.50 | 0.26 | 0.77 | 0.61 | 0.6567153 |
| Adjusted | 0.07 | 0.03 | 0.19 | 0.41 | 0.13 | 0.73 | 0.54 | 0.59314406 |
| R Square | | | | | | | | |
| Standard | 0.26 | 0.18 | 0.01 | 0.20 | 3.69E−03 | 0.07 | 2.76E−03 | 0.00739343 |
| Error | | | | | | | | |
| RMSE | 0.23 | 0.16 | 0.01 | 0.18 | 3.34E−03 | 0.06 | 2.50E−03 | 0.01180409 |
| Obser- | 32 | 32 | 31 | 33 | 33 | 33 | 33 | 33 |
| vations | | | | | | | | |

TABLE 6
Correlation Matrix for Atlas Scientific Sensors

| | DO 97 (mg/L) | pH 99 ( ) | EC 100 (uS) | RTD 102 (c) | ORP 98 (mV) |
|---|---|---|---|---|---|
| DO 97 (mg/L) | 1 | — | — | — | — |
| pH 99 ( ) | −0.1004527 | 1 | — | — | — |
| EC 100 (uS) | −0.3146643 | 0.58183886 | 1 | — | — |
| RTD 102 (c) | 0.27497674 | 0.34699723 | 0.17328755 | 1 | — |
| ORP 98 (mV) | 0.38292437 | −0.6047262 | −0.4330551 | 0.06550297 | 1 |

TABLE 7
Turbidity Example Determination

| | | Sensor | Coefficient* |
|---|---|---|---|
| Parameter | Coefficient | Value | Sensor Value |
| Intercept | 0.104391036 | 1 | 0.10439104 |
| Dissolved Oxygen | −0.02176488 | 9 | −0.19588391 |
| Hydrogen | 6349534.248 | 3.0903E−08 | 0.19621937 |
| Electrical Conductivity | 0.000155723 | 539.35 | 0.08398944 |
| Temperature | 0.011084577 | 22.15 | 0.24552338 |
| Oxidation Reduction | 0.000192205 | 595.06 | 0.11437352 |
| Potential | | | |
| Sum to Obtain | — | — | 0.54861284 |
| Predicted Value* | | | |
| Measured Value* | — | — | 0.58 |

*NTU

TABLE 8
Copper Example Estimation

| | | Sensor | Coefficient* |
|---|---|---|---|
| Parameter | Coefficient | Value | Sensor Value |
| Intercept | 4.306967881 | 1 | 4.30696788 |
| Dissolved Oxygen | 0.019235283 | 7.74 | 0.14888109 |
| Hydrogen | 1725212.982 | 1.1482E−08 | 0.0198081 |
| Electrical | −0.002977988 | 558.25 | −1.66246195 |
| Conductivity | | | |
| Temperature | −0.103304748 | 21.99 | −2.27167142 |
| Oxidation Reduction | 0.000171739 | 313 | 0.05375441 |
| Potential | | | |
| Sum to Obtain | | | 0.59527811 |
| Predicted Value* | | | |
| Measured Value* | | | 0.604 |

*mg/L

TABLE 9
DOC Example Estimation

| | | | Coefficient* |
|---|---|---|---|
| Parameter | Coefficient | Sensor Value | Sensor Value |
| Intercept | 1.85515231 | 1 | 1.85515231 |
| Dissolved Oxygen | −0.073072534 | 8.72 | −0.6371925 |
| Hydrogen | 8519314.701 | 1.23027E−08 | 0.10481047 |
| Electrical | 0.000621324 | 563.65 | 0.35020932 |
| Conductivity | | | |
| Temperature | 0.039508064 | 22.84 | 0.90236418 |
| Oxidation Reduction | 0.000466799 | 418.08 | 0.19515919 |
| Potential | | | |
| Sum to Obtain | — | — | 2.77050296 |
| Predicted Value* | | | |
| Measured Value* | — | — | 2.805 |

*mg/L

TABLE 10
Buffer Calibration Test Strip Results

| | | | | | Grayscale | | | |
|---|---|---|---|---|---|---|---|---|
| Brand | pH | R | G | B | Intensity | L | A | B |
| 16in1A | 4 | 244 | 220 | 47 | 207.45 | 87.30 | −8.36 | 79.68 |
| 16in1A | 7 | 244 | 142 | 71 | 164.40 | 68.78 | 33.08 | 53.13 |
| 16in1A | 10 | 252 | 35 | 198 | 118.47 | 58.02 | 87.10 | −32.38 |

TABLE 11
Tap Water Testing returncolors.py Results

| | | | | | Grayscale | | | |
|---|---|---|---|---|---|---|---|---|
| Brand | pH | R | G | B | Intensity | L | A | B |
| 16in1A | 7.4 | 242 | 157 | 60 | 171.357 | 71.77 | 23.93 | 61.01 |

TABLE 12
Tap Water Testing Best Match Calculations

| | Gradient RGB | | | Gradient LAB | | | Best Match | |
|---|---|---|---|---|---|---|---|---|
| pH | R | G | B | L | A | B | RGB | LAB |
| 6 | 243 | 171 | 51 | 74.04 | 17.91 | 68.39 | 0.96903 | 9.789475908 |
| 6.1 | 243 | 168 | 53 | 73.4683 | 19.37 | 67.1402 | 0.97447 | 7.825551206 |
| 6.2 | 243 | 166 | 54 | 72.9052 | 20.844 | 65.8248 | 0.98009 | 5.82943873 |
| 6.3 | 243 | 163 | 56 | 72.3507 | 22.332 | 64.4438 | 0.98589 | 3.830881455 |
| 6.4 | 243 | 160 | 58 | 71.8048 | 23.834 | 62.9972 | 0.99186 | 1.989744588 |
| 6.5 | 243 | 157 | 60 | 71.2675 | 25.35 | 61.485 | 0.99802 | 1.5811482 |
| 6.6 | 243 | 154 | 62 | 70.7388 | 26.88 | 59.9072 | 0.99216 | 3.315616426 |
| 6.7 | 243 | 151 | 64 | 70.2187 | 28.424 | 58.2638 | 0.98539 | 5.49192067 |
| 6.8 | 244 | 148 | 66 | 69.7072 | 29.982 | 56.5548 | 0.97841 | 7.794444931 |
| 6.9 | 244 | 145 | 68 | 69.2043 | 31.554 | 54.7802 | 0.97124 | 10.17580418 |
| 7 | 244 | 142 | 71 | 68.71 | 33.14 | 52.94 | 0.96386 | 12.62325752 |

### Tables 13-15

### (See Appendix A—Computer Program Listing)

What is claimed is:

1. A water filtration apparatus, comprising:
(a) a water filtration loop in fluid communication between a fluid reservoir for receiving water at an inlet, and an outlet;
(b) at least one water filter connected within said water filtration loop;
(c) valves connected along said water filtration loop to control the flow of water through the filters;
(d) a controller configured for controlling the on/off state of said valves when determining the condition of the water filters;
(e) wherein said valves can be switched between a first state in which water bypasses said at least one water filter in traversing from inlet to outlet, and a second state in which water passes through said at least one water filter;
(f) a plurality of water quality sensors on said controller for collecting water quality information, comprising: a flow sensor, an oxidation-reduction potential (ORP) sensor, dissolved oxygen (DO) sensor, a pH sensor. an electrical conductivity (EC) sensor, and a temperature sensor; and
(g) wherein said controller executes firmware instructions from its microcontroller, for performing steps comprising:
(i) switching said valves between said first state and said second state, or detecting that manual or automated switching has occurred between said first state and said second state, whereby said controller is configured to collect and assess water quality comprising steps of:
comparing the quality of water which passes through the filters with water that has not passed through the filters to determine the replacement interval of the filter media as determined by the combination of sensor values;
comparing the quality of water which passes through the filters with water that has not passed through the filters to determine the efficacy of the filter media at removing specific contaminants present in the water as determined by the combination of sensor values and comparison to stored reference values;
comparing the quality of water which passes through the filters with water that has not passed through the filters to determine the type of contaminants present in the water by comparing the combination of sensor values to stored reference values; and
comparing the quality of water which has not passed through the filters to measurements from other devices located in the same water system to determine the source of contamination in the water.

2. The apparatus of claim 1, wherein said at least one water filter is selected from the group of filter types consisting of reverse osmosis, kinetic degradation, sediment, remineralization, and granular activated carbon filters.

3. The apparatus of claim 1, further comprising a recycle flow path in the water system, whereby water may be redirected from the outlet back to the fluid reservoir for receiving water at the inlet.

4. The apparatus of claim 1, further comprising a communications circuit coupled to said microcontroller, to allow said controller to communicate with external devices and/or displays.

5. The apparatus of claim 1, further comprising a communications circuit coupled to said microcontroller, to allow said controller to store collected information from each test session into a repository allowing access by collaborators.

6. The apparatus of claim 1, wherein said controller further comprises analog conditioning circuitry for one or more of the sensors.

7. The apparatus of claim 1, wherein said valves are coupled to an actuator allowing said controller to switch these valves automatically in performing the filter testing.

8. The apparatus of claim 1, wherein said valves are manual valves.

9. The apparatus of claim 8, wherein a valve position sensing device is coupled to one or more of said valves to communicate the state of said one or more valves back to the controller, allowing said controller to automatically perform its operations based on positioning of the valves.

10. The apparatus of claim 1, further comprising calibrating the sensors prior to collection of water samples.

11. A water filtration apparatus, comprising:
(a) a water filtration loop in fluid communication between a fluid reservoir for receiving water at an inlet, and an outlet;
(b) at least one water filter connected within said water filtration loop;
(c) valves connected along said water filtration loop to control the flow of water through the filters, wherein each said valve is a manual valve coupled to a position sensing device to communicate the state of said one or more valves back to the controller, allowing said controller to automatically perform its operations based on positioning of the valves;
(d) a controller configured for controlling the on/off state of said valves when determining the condition of the water filters;
(e) wherein said valves can be switched between a first state in which water bypasses said at least one water filter in traversing from inlet to outlet, and a second state in which water passes through said at least one water filter;
(f) a plurality of water quality sensors on said controller for collecting water quality information, comprising: a flow sensor, an oxidation-reduction potential (ORP) sensor, dissolved oxygen (DO) sensor, a pH sensor. an electrical conductivity (EC) sensor, and a temperature sensor; and
(g) wherein said controller executes firmware instructions from its microcontroller, for performing steps comprising:
(i) responding to the detection of said valves being switched between said first state and said second state, said controller is configured to collect and assess water quality comprising steps of:
comparing the quality of water which passes through the filters with water that has not passed through the filters to determine the replacement interval of the filter media as determined by the combination of sensor values;
comparing the quality of water which passes through the filters with water that has not passed through the filters to determine the efficacy of the filter media at removing specific contaminants present in the water as determined by the combination of sensor values and comparison to stored reference values;
comparing the quality of water which passes through the filters with water that has not passed through the filters to determine the type of contaminants present in the water by comparing the combination of sensor values to stored reference values; and
comparing the quality of water which has not passed through the filters to measurements from other devices located in the same water system to determine the source of contamination in the water.

12. The apparatus of claim 11, wherein said at least one water filter is selected from the group of filter types consisting of reverse osmosis, kinetic degradation, sediment, remineralization, and granular activated carbon filters.

13. The apparatus of claim 11, further comprising a recycle flow path in the water system, whereby water may be redirected from the outlet back to the fluid reservoir for receiving water at the inlet.

14. The apparatus of claim 11, further comprising a communications circuit coupled to said microcontroller, to allow said controller to communicate with external devices and/or displays.

15. The apparatus of claim 11, further comprising a communications circuit coupled to said microcontroller, to allow said controller to store collected information from each test session into a repository allowing access by collaborators.

16. A water filtration apparatus, comprising:
(a) a water filtration loop in fluid communication between a fluid reservoir for receiving water at an inlet, and an outlet;
(b) at least one water filter connected within said water filtration loop;
(c) valves connected along said water filtration loop to control the flow of water through the filters, wherein said valves are coupled to an actuator coupled to said controller which switches these valves automatically in performing filter testing;
(d) a controller configured for controlling the on/off state of said valves when determining the condition of the water filters;
(e) wherein said valves are switched between a first state in which water bypasses said at least one water filter in traversing from inlet to outlet, and a second state in which water passes through said at least one water filter;
(f) a plurality of water quality sensors on said controller for collecting water quality information, comprising: a flow sensor, an oxidation-reduction potential (ORP) sensor, dissolved oxygen (DO) sensor, a pH sensor. an electrical conductivity (EC) sensor, and a temperature sensor; and
(g) wherein said controller executes firmware instructions from its microcontroller, for performing steps comprising:
(i) switching said valves between said first state and said second state, upon which said controller is configured to collect and assess water quality comprising steps of:
comparing the quality of water which passes through the filters with water that has not passed through the filters to determine the replacement interval of the filter media as determined by the combination of sensor values;
comparing the quality of water which passes through the filters with water that has not passed through the filters to determine the efficacy of the filter media at removing specific contaminants present in the water as determined by the combination of sensor values and comparison to stored reference values;
comparing the quality of water which passes through the filters with water that has not passed through the filters to determine the type of contaminants present in the water by comparing the combination of sensor values to stored reference values; and
comparing the quality of water which has not passed through the filters to measurements from other devices located in the same water system to determine the source of contamination in the water.

17. The apparatus of claim 16, wherein said at least one water filter is selected from the group of filter types consisting of reverse osmosis, kinetic degradation, sediment, remineralization, and granular activated carbon filters.

18. The apparatus of claim 16, further comprising a recycle flow path in the water system, whereby water may be redirected from the outlet back to the fluid reservoir for receiving water at the inlet.

19. The apparatus of claim 16, further comprising a communications circuit coupled to said microcontroller, to allow said controller to communicate with external devices and/or displays.

20. The apparatus of claim 16, further comprising a communications circuit coupled to said microcontroller, to allow said controller to store collected information from each test session into a repository allowing access by collaborators.
